(19) United States
(12) Patent Application Publication
(10) Pub. No.: US 2020/0135309 A1
(43) Pub. Date: Apr. 30, 2020

(54) CLINICAL DASHBOARD FOR MEDICAL DEVICE

(71) Applicant: Physio-Control, Inc., Redmond, WA (US)

(72) Inventors: Randy L. Merry, Woodinville, WA (US); Ken Peterson, Bellevue, WA (US); Mitchell A. Smith, Sammamish, WA (US); Dana S. Lewis, Woodinville, WA (US); Gerard Verkerk, Seattle, WA (US); James Wootten, Kirkland, WA (US)

(21) Appl. No.: 16/733,156

(22) Filed: Jan. 2, 2020

Related U.S. Application Data
(63) Continuation of application No. 13/836,304, filed on Mar. 15, 2013, now abandoned.
(60) Provisional application No. 61/667,246, filed on Jul. 2, 2012.

(51) Int. Cl. G16H 10/60; G16H 40/63

(52) U.S. Cl. CPC G16H 10/60; G16H 40/63

(57) ABSTRACT

A clinical dashboard device, system, method for providing clinical data for clinical dashboard display on a device is disclosed. The system includes a server for providing clinical dashboard data, which may include data from one or more defibrillators, and further includes a clinical dashboard device. The clinical dashboard device includes a processor, a communication module configured for enabling communication between the processor and the server for receiving the clinical dashboard data from the server, and a clinical dashboard client. The clinical dashboard client includes a clinical dashboard data manager module including an instance of a clinical dashboard data manager service that provides managed clinical dashboard data between one or more clinical dashboard applications and the server. The instance of the clinical dashboard data manager service further provides managed display of managed clinical dashboard data on the display of the clinical dashboard device. The processor is configured to execute the instance.

### CROSS REFERENCE TO RELATED PATENT APPLICATIONS

This patent application may be found to be related to U.S.A. patent application Ser. No. ______, entitled “Decision Support Tool For Use With a Medical Monitor-Defibrillator”, filed contemporaneously herewith in the name of Ken Peterson et al.; and U.S.A. patent application Ser. No. ______ entitled “Medical Monitor-Defibrillator with Defibrillator and Data Operations Processors”, filed contemporaneously herewith in the name of Ken Peterson et al.

### FIELD

This invention generally relates to external defibrillators.

### BACKGROUND

In humans, the heart beats to sustain life. In normal operation, it pumps blood through the various parts of the body. More particularly, the various chamber of the heart contract and expand in a periodic and coordinated fashion, which causes the blood to be pumped regularly. More specifically, the right atrium sends deoxygenated blood into the right ventricle. The right ventricle pumps the blood to the lungs, where it becomes oxygenated, and from where it returns to the left atrium. The left atrium pumps the oxygenated blood to the left ventricle. The left ventricle, then, expels the blood, forcing it to circulate to the various parts of the body and from where it returns to the right atrium to start the oxygenation-deoxygenation cycle of the blood all over again.

The heart chambers pump because of the heart's electrical control system. More particularly, the sinoatrial (SA) node generates an electrical impulse, which generates further electrical signals. These further signals cause the above-described contractions of the various chambers in the heart to occur in the correct sequence. The electrical pattern created by the sinoatrial (SA) node is called a sinus rhythm.

Sometimes, however, the electrical control system of the heart malfunctions, which can cause the heart to beat irregularly, or not at all. The cardiac rhythm is then generally called an arrhythmia. Arrhythmias may be caused by electrical activity from locations in the heart other than the SA node. Some types of arrhythmia may result in inadequate blood flow, thus reducing the amount of blood pumped to the various parts of the body. Some arrhythmias may even result in a Sudden Cardiac Arrest (SCA). In an SCA, the heart fails to pump blood effectively, and, if not corrected, can result in death. It is estimated that SCA results in more than 250,000 deaths per year in the United States alone. Further, an SCA may result from a condition other than an arrhythmia.

One type of arrhythmia associated with SCA is known as Ventricular Fibrillation (VF). VF is a type of malfunction where the ventricles make rapid, uncoordinated movements, instead of the normal contractions. When that happens, the heart does not pump enough blood to deliver enough oxygen to the vital organs. The person's condition will deteriorate rapidly and, if not corrected in time, will result in death, e.g. within ten minutes.

Ventricular Fibrillation can often be reversed using a life-saving device called a defibrillator. A defibrillator, if applied properly, can administer an electrical shock to the heart. The shock may terminate the VF, thus giving the heart the opportunity to resume normal contractions in pumping blood. If VF is not terminated, the shock may be repeated, often at escalating energies.

A challenge with defibrillation is that the electrical shock must be administered very soon after the onset of VF. There is not much time to do this since the survival rate of persons suffering from VF decreases by about 10% for each minute the administration of a defibrillation shock is delayed. After about 10 minutes the rate of survival for SCA victims averages less than 2%.

The challenge of defibrillating early after the onset of VF is being met in a number of ways. First, for some people who are considered to be at a higher risk of VF or other heart arrythmias, an Implantable Cardioverter Defibrillator (ICD) can be implanted surgically. An ICD can monitor the person's heart, and administer an electrical shock as needed. As such, an ICD reduces the need to have the higher-risk person be monitored constantly by medical personnel.

Regardless, VF can occur unpredictably, even to a person who is not considered at risk. As such, VF can be experienced by many people who lack the benefit of ICD therapy. When VF occurs to a person who does not have an ICD, they collapse, because the blood flow has stopped. They should receive therapy quickly after the onset of VF or they will die.

For a VF victim without an ICD, a different type of defibrillator can be used, which is called an external defibrillator. External defibrillators have been made portable, so they can be brought to a potential VF victim quickly enough to revive them.

During VF, the person's condition deteriorates because the blood is not flowing to the brain, heart, lungs, and other organs. The blood flow must be restored, if resuscitation attempts are to be successful.

Cardiopulmonary Resuscitation (CPR) is one method of forcing blood to again flow in a person experiencing cardiac arrest. In addition, CPR is the primary recommended treatment for some patients with some kinds of non-VF cardiac arrest, such as asystole and pulseless electrical activity (PEA). CPR is a combination of techniques that include chest compressions to force blood circulation, and rescue breathing to force respiration.

Properly administered CPR provides oxygenated blood to critical organs of a person in cardiac arrest, thereby minimizing the deterioration that would otherwise occur. As such, CPR can be beneficial for persons experiencing VF, because it slows down the deterioration that would otherwise occur while a defibrillator is being retrieved. For patients with an extended down-time, survival rates are higher if CPR is administered prior to defibrillation.

Advanced medical devices may be used to assist the CPR process by coaching a rescuer who performs CPR. For example, a medical device can issue instructions, and even prompts, for the rescuer to perform CPR more effectively.

While some advanced medical devices provide coaching, defibrillator operators may benefit from additional coaching.

### BRIEF SUMMARY

The present description gives instances of devices, systems, software and methods, the use of which may help overcome problems and limitations of the prior art.

More specifically, a clinical dashboard device, system, method for providing clinical data for clinical dashboard display on a device is disclosed. The system includes a server for providing clinical dashboard data, which may include data from one or more defibrillators, and further includes a clinical dashboard device. The clinical dashboard device includes a processor, a communication module configured for enabling communication between the processor and the server for receiving the clinical dashboard data from the server, and a clinical dashboard client. The clinical dashboard client includes a clinical dashboard data manager module including an instance of a clinical dashboard data manager service that provides managed clinical dashboard data between one or more clinical dashboard applications and the server. The instance of the clinical dashboard data manager service further provides managed display of managed clinical dashboard data on the display of the clinical dashboard device. The processor is configured to execute the instance.

These and other features and advantages of this description will become more readily apparent from the following Detailed Description, which proceeds with reference to the drawings, in which:

### BRIEF DESCRIPTION OF THE DRAWINGS

FIG. 1 is an illustrative diagram of a scene showing the use of an external defibrillator to save the life of a person according to this disclosure.

FIG. 2 is a table listing two illustrative types of the external defibrillator shown in FIG. 1, and who they might be used by.

FIG. 3 is a diagram showing components of an external defibrillator, such as the one shown in FIG. 1, configured in an illustrative embodiment according to this disclosure.

FIGS. 4A, 4B show functional diagrams of illustrative systems for providing clinical dashboard data to a caregiver according to this disclosure.

FIG. 5 is an enlarged view of one illustrative system including a server, a web server, and a defibrillator.

FIG. 6 is an enlarged view of an illustrative clinical dashboard device of the system in FIG. 4.

FIG. 7 shows an illustrative dashboard generator of this disclosure

FIGS. 8 and 9 are an enlarged view of another illustrative clinical dashboard device and server of the system in FIG. 4.

FIG. 10 is a flowchart for the generation of a clinical dashboard according to this disclosure.

FIGS. 11-27 show illustrative clinical dashboards configurable using the teachings of this disclosure.

### DETAILED DESCRIPTION

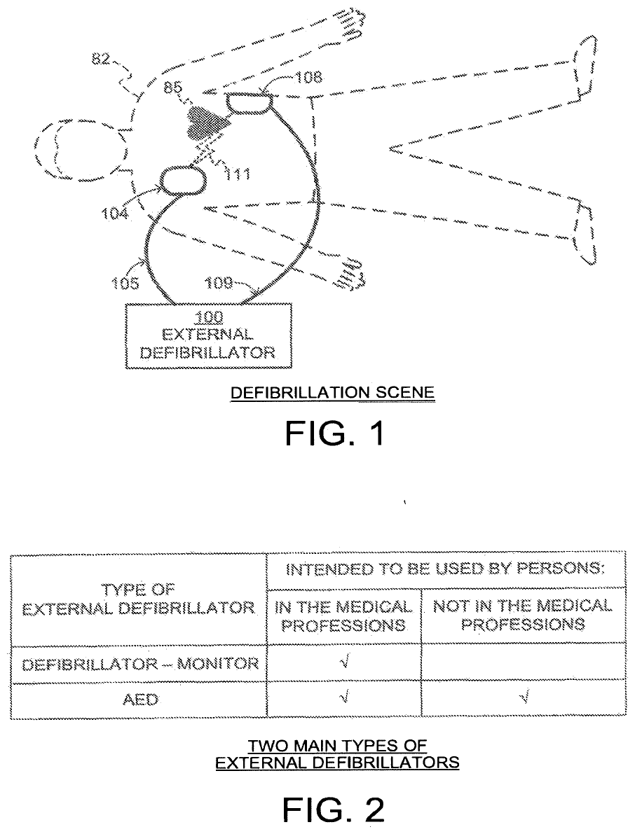

FIG. 1 is a diagram of a defibrillation scene showing the use of an external defibrillator to save the life of a person according to this disclosure. As shown, a person 82 is lying on his back. Person 82 could be a patient in a hospital, or someone found unconscious, and then turned over onto his back. Person 82 is experiencing a condition in their heart 85, which could be Ventricular Fibrillation (VF).

A portable external defibrillator 100 has been brought close to person 82. At least two defibrillation electrodes 104, 108 are typically provided with external defibrillator 100, and are sometimes called electrodes 104, 108. Electrodes 104, 108 are coupled together with external defibrillator 100 via respective electrode leads 105, 109. A rescuer (not shown) has attached electrodes 104, 108 to the skin of person 82. Defibrillator 100 is administering, via electrodes 104, 108, a brief, strong electric pulse 111 through the body of person 82. Pulse 111, also known as a defibrillation shock, also goes through heart 85, in an attempt to restart it, for saving the life of person 82. Defibrillator 100 can be one of different types, each with different sets of features and capabilities. The set of capabilities of defibrillator 100 is determined based upon who would use it and what training they would be likely to have. Examples are now described.

FIG. 2 is a table listing two typical types of external defibrillators, and who they are primarily intended to be used by. A first type of defibrillator 100 is generally called a defibrillator-monitor, because the defibrillator part is typically formed as a single unit with a patient monitor part. A defibrillator-monitor is sometimes called monitor-defibrillator. A defibrillator-monitor is intended to be used by persons in the medical profession, such as doctors, nurses, paramedics, emergency medical technicians, etc. who may be trained to provide medical treatment to the patient during a defibrillation process based upon information provided by the monitor. Such a defibrillator-monitor is intended to be used in a pre-hospital or hospital scenario.

The defibrillator part may be dedicated to a particular mode of operation. Alternatively, the defibrillator part may be configured to operate in more than one modes of operation. One mode of operation of the defibrillator part may be that of an automated defibrillator, which can determine whether a shock is needed and, if so, charge to a predetermined energy level and instruct the user to administer the shock. Another mode of operation may be that of a manual defibrillator, where the user determines the need and controls administering the shock. In this embodiment, one illustrative defibrillator is configured to enable both automated defibrillation and manual defibrillation modes of operation depending upon the selection of the user. As a patient monitor, the device has features additional to what is minimally needed for mere operation as a defibrillator. These features can be for monitoring physiological indicators of a person in an emergency scenario. These physiological indicators are typically monitored as signals. For example, these signals can include a person's full ECG (electrocardiogram) signals, or impedance between two electrodes. Additionally, these signals can be about the person's temperature, non-invasive blood pressure (NIBP), arterial oxygen saturation/pulse oximetry (SpO2), the concentration or partial pressure of carbon dioxide in the respiratory gases, which is also known as capnography, and so on. These signals can be further stored and/or transmitted as patient data.

A second type of external defibrillator 100 is generally called an AED, which stands for “Automated External Defibrillator”. An AED typically makes the shock/no shock determination by itself, automatically. Indeed, it can sense enough physiological conditions of the person 82 via only the shown defibrillation electrodes 104, 108 of FIG. 1. In its present embodiments, an AED can either administer the shock automatically, or instruct the user to do so, e.g. by pushing a button. Being of a much simpler construction, an AED typically costs much less than a defibrillator-monitor. As such, it makes sense for a hospital, for example, to deploy AEDs at its various floors, in case the more expensive defibrillator-monitor is more critically being deployed at an Intensive Care Unit, and so on.

AEDs, however, can also be used by people who are not trained in the medical profession. More particularly, an AED can be used by many professional first responders, such as policemen, firemen, etc. Even a person with only first-aid training can use one. And AEDs increasingly can supply instructions to whoever is using them.

AEDs are thus particularly useful, because it is so critical to respond quickly, when a person suffers from VF. Often, the people who will first reach the VF sufferer may not be in the medical profession.

Increasing awareness of the short survival time of a patient experiencing a VF, has resulted in AEDs being deployed more pervasively in public or semi-public spaces, enabling members of the public to use one provided they have obtained first aid and CPR/AED training. In this way, defibrillation can be administered sooner after the onset of VF, to hopefully be effective in rescuing the person.

There are additional types of external defibrillators, which are not listed in FIG. 2. For example, a hybrid defibrillator can have aspects of an AED, and also of a defibrillator-monitor. An illustrative example may be an AED provided with an ECG monitoring capability.

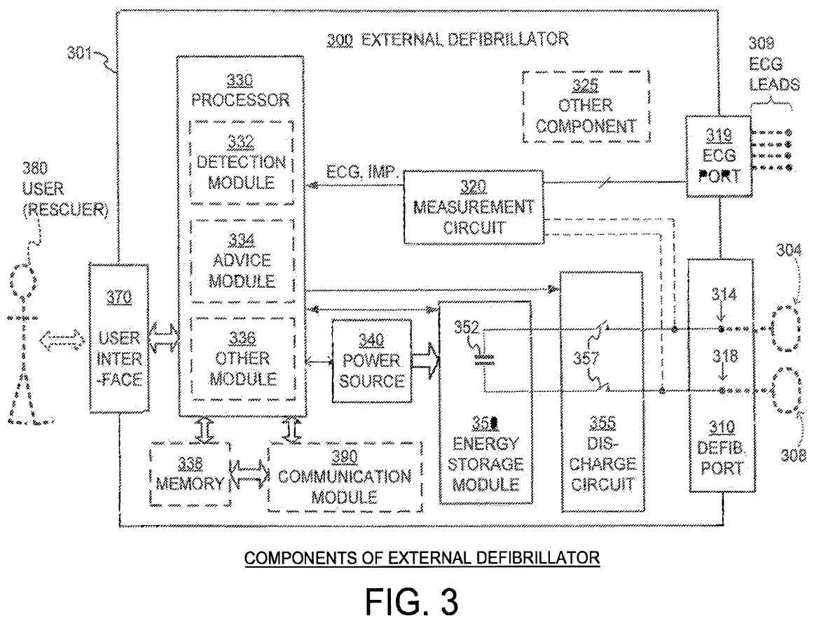

FIG. 3 is a diagram showing components of an external defibrillator 300 configured in an illustrative embodiment according to this disclosure. These components can be configured, for example, in external defibrillator 100 of FIG. 1. Plus, these components of FIG. 3 can be provided in a housing 301, which is also known as casing 301.

External defibrillator 300 is intended for use by a user 380, who would be the rescuer. Defibrillator 300 typically includes a defibrillation port 310, which may be configured as a socket (not shown) in housing 301. Defibrillation port 310 includes nodes 314, 318. Defibrillation electrodes 304, 308, which can be similar to electrodes 104, 108 in FIG. 1, can be plugged into defibrillation port 310, so as to make electrical contact with nodes 314, 318, respectively. It is also possible that electrodes can be hard-wired to defibrillation port 310, etc. Either way, defibrillation port 310 can be used for guiding to person 82 via electrodes an electrical charge that has been stored in defibrillator 300, as discussed below.

If defibrillator 300 is actually a defibrillator-monitor, as was described with reference to FIG. 2, then it will typically also have an ECG port 319 in housing 301, for plugging in ECG leads 309. ECG leads 309 can help sense an ECG signal, e.g. a 12-lead signal, or a signal taken from a different number of leads. Moreover, a defibrillator-monitor could have additional ports (not shown), and another component 325 for the above described additional features, such as for receipt of patient signals.

Defibrillator 300 also includes a measurement circuit 320. Measurement circuit 320 receives physiological signals from ECG port 319, and also from other ports, if provided. These physiological signals are sensed, and information about them is rendered by circuit 320 as data, or other signals, etc.

If defibrillator 300 is actually an AED, it may lack ECG port 319. Measurement circuit 320 can obtain physiological signals in this case through nodes 314, 318 instead, when defibrillation electrodes 304, 308 are attached to person 82. In these cases, a person's ECG signal can be sensed as a voltage difference between electrodes 304, 308. Plus, impedance between electrodes 304, 308 can be sensed for detecting, among other things, whether these electrodes 304, 308 have been inadvertently disconnected from the person.

Defibrillator 300 also includes a processor 330. Processor 330 may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 330 may include a number of modules. One such module can be a detection module 332, which senses outputs of measurement circuit 320. Detection module 332 can include a VF detector. Thus, the person's sensed ECG can be used to determine whether the person is experiencing VF.

Another such module in processor 330 can be an advice module 334, which arrives at a piece of instructional advice based on outputs of detection module 332. Advice module 334 can include a Shock Advisory Algorithm residing in a memory unit (not shown) in the advice module for instructing the processor to implement decision rules, etc. Alternatively, the Shock Advisory Algorithm may reside in part or in whole on a memory 338 of the defibrillator. The instruction to the processor can be to shock, to not shock, to administer other forms of therapy, and so on. If the instruction to the processor is to shock, in some external defibrillator embodiments, the processor is configured to report that instruction to the user via user interface 370, and to prompt the user to do it. In other embodiments, the processor may be configured to execute the instructional advice, by administering the shock. If the instructional advice is to administer CPR, the processor may be configured to enable defibrillator 300 to issue prompts to administer CPR, etc.

Processor 330 can include additional modules, such as module 336, for other functions. In addition, if other component 325 is provided, it may be operated in part by processor 330 or by another processor.

Defibrillator 300 optionally further includes the memory 338, which can work together with processor 330. Memory 338 may be implemented in any number of ways. Such ways include, by way of example and not of limitation, nonvolatile memories (NVM), read-only memories (ROM), random access memories (RAM), any combination of these, etc.. Memory 338, if provided, may include programs containing instructions for execution by processor 330 or other processors that may be included in the external defibrillator. The programs provide instructions for execution by the processor 330, and can also include instructions regarding protocols and decision making analytics, etc. that can be used by advice module 334. In addition, memory 338 can store prompts for user 380, etc. Moreover, memory 338 can store patient data.

Defibrillator 300 may also include a power source 340. To enable portability of defibrillator 300, power source 340 typically includes a battery. Such a battery is typically implemented as a battery pack, which can be rechargeable or not. Sometimes, a combination is used, of rechargeable and non-rechargeable battery packs. Other embodiments of power source 340 can include an AC power override, whereby AC power, instead of power from power source 340 is delivered to an energy storage module 350 when AC power is available. In some embodiments, power source 340 is controlled by processor 330.

Defibrillator 300 additionally includes the energy storage module 350. Module 350 is where electrical energy is stored in preparation for a sudden discharge to administer a shock. The charge to module 350 from power source 340 to the right amount of energy can be controlled by processor 330. In typical implementations, module 350 includes one or more capacitors 352, and may include other circuitry.

Defibrillator 300 moreover includes a discharge circuit 355. Circuit 355 can be controlled to permit the energy stored in module 350 to be discharged to nodes 314, 318, and thus also to defibrillation electrodes 304, 308. Circuit 355 can include one or more switches 357. Those can be made in a number of ways, such as by an H-bridge, and in other ways well known in the art.

Defibrillator 300 further includes the user interface 370 for user 380. User interface 370 can be made in any number of ways. For example, interface 370 may include a screen, to display a parameter of a patient that is detected and measured, provide visual feedback to the rescuer for their resuscitation attempts, and so on. Interface 370 may also include a speaker, to issue voice prompts, etc. Interface 370 may additionally include various controls, such as pushbuttons, keyboards, and so on. In addition, discharge circuit 355 can be controlled by processor 330, or directly by user 380 via user interface 370, and so on.

Defibrillator 300 can optionally include other components. For example, a communication module 390 may be provided for communicating with other devices. Such communication can be performed wirelessly, or via wire, or by infrared communication, and so on. In this way, data can be communicated from the defibrillator 300 to external devices, such as patient data, incident information, therapy attempted, CPR performance, and so on.

Having thus introduced background on the general operation of a defibrillator, we now turn to features that are provided by this disclosure.

A clinical dashboard device, system, method for providing clinical data for clinical dashboard display on a device is disclosed. The system includes a server for providing clinical dashboard data, which may include data from one or more defibrillators, and further includes a clinical dashboard device. The clinical dashboard device includes a processor, a communication module configured for enabling communication between the processor and the server for receiving the clinical dashboard data from the server, and a clinical dashboard client. The clinical dashboard client includes a clinical dashboard data manager module including an instance of a clinical dashboard data manager service that provides managed clinical dashboard data between one or more clinical dashboard applications and the server. The instance of the clinical dashboard data manager service further provides managed display of managed clinical dashboard data on the display of the clinical dashboard device. The processor is configured to execute the instance.

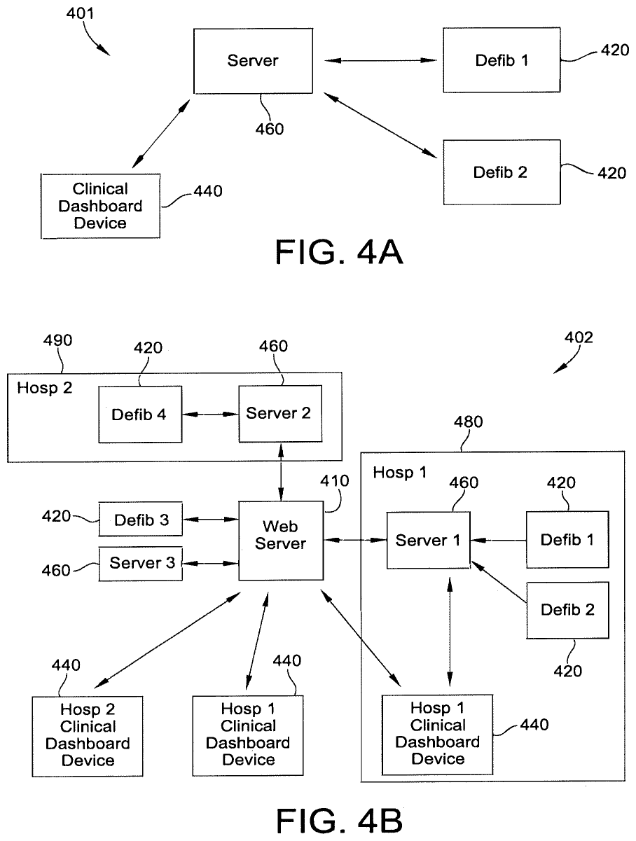

FIGS. 4A, 4B show illustrative functional diagrams of illustrative systems 401, 402 for providing clinical dashboard data to a caregiver according to this disclosure. The system 401 shown in FIG. 4A comprises a server 460, one or more defibrillators illustratively shown as defibrillators 420, and one or more clinical dashboard devices illustratively shown as clinical dashboard devices 440. The system shown in FIG. 4B comprises a web server 410, one or more servers 460 (three servers denoted server 1, server 2, and server 3 are shown), and one or more defibrillators 420 (four defibrillators denoted defibrillator 1, defibrillator 2, defibrillator 3, and defibrillator 4 are shown). As explained below, the delivery and display of clinical data residing on servers throughout the network for clinical dashboard display on defibrillators throughout the network may be advantageously configured through protocols, rules, etc., by clinical dashboard devices in the network and managed by a clinical dashboard client in the defibrillators. For example, the clinical dashboard device illustratively may serve as the editor for configuring the client; programming the client in the defibrillator with the proper protocol, sequences, rules, configurations, etc. for the client to use in managing the clinical display. The clinical dashboard device may further configure the clinical data residing on server 460 with protocols, rules, etc. for delivery and display on the clinical dashboard according to this disclosure.

The system 401 of FIG. 4A may be a local area network of a hospital, for example. In this example, the clinical dashboard device 440 may configure a client residing on the defibrillators 420 and clinical data residing on server 460 with protocols, rules, etc. for delivery and display on a clinical dashboard on defibrillators 420 (e.g., defibrillator 1 and defibrillator 2) according to this disclosure. The client on the defibrillator is used to manage the dashboard display according to this disclosure.

In FIG. 4B, the local area network of system 401 (shown as hospital 1 in FIG. 4B and denoted by the number 480) is connected through a web server 410 to an internetworking system 402 shown in FIG. 4B. In this system 402, a second local area network 490 (shown as hospital 2 in FIG. 4B) is also connected to the web server 410. The web server 410 is also shown to be in communication with one defibrillator 420 (denoted defibrillator 3), one server (denoted server 3) and two clinical dashboard devices 440 (denoted as hospital 1 clinical dashboard device and hospital 2 clinical dashboard device) that are outside of either local area networks 480, 490. Another advantage of this disclosure is that for a given set of clinical dashboards, servers, and defibrillators that have been associated to each other through rules, permissions, etc. according to this disclosure, no matter where the clinical dashboard device, the defibrillator, or the server of the associated set may be on the internetwork 402, the clinical dashboard display has clinical data on associated servers configured by the associated clinical dashboard device and managed by the client on the defibrillator to provide a more robust, cohesive, and enhanced display to the user.

While FIGS. 4A, 4B and elsewhere there is discussion of illustrative embodiments using a defibrillator, it will be appreciated that the teachings of this disclosure may illustratively also be used with other medical devices that are used for coaching a caregiver through a treatment.

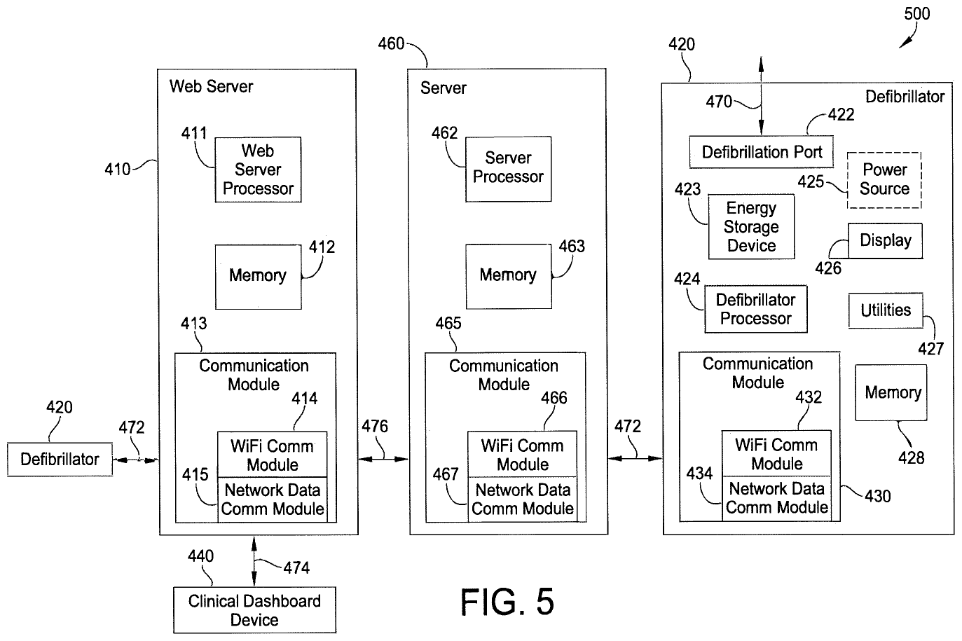

FIG. 5 shows an enlarged view of one illustrative system 500 according to this disclosure including a server 410, a web server 410, and a defibrillator 420, and a clinical dashboard device 440. As shown in FIG. 5, defibrillator 420 comprises an energy storage device 423 for storing an electrical charge; a defibrillation port 422; a display 426; a defibrillator processor 424 configured to control the display and when an electrical charge is applied to the defibrillation port 422 for defibrillating a patient; a memory 428, and a power source 424. Defibrillator 420 and its various components, can be as already described with reference to FIG. 3 above.

The display 426 of the defibrillator 410 may be a visual display capable of displaying data transmitted from defibrillator processor. Display 426 for use with this disclosure may include an LCD screen, an e-paper display, or other bi-stable display, a CRT display or any other type of visual display.

Communication module 430 is hardware and software configured to transmit data to and from the defibrillator 410. Illustratively, the communication module 430 is configured to transmit data from the defibrillator to the web server 410. The web server may be a computer, a laptop, a server, a mobile computing device, or other computing device. Alternatively, the defibrillator processor 424 may receive data from the web server through the communication module 430 to the defibrillator 420. Hence, communication module 430 provides for the bidirectional transmission of data between the defibrillator 410 and the web server 410. In order to allow for the bidirectional flow of data between the defibrillator and the external utility, the web server is likewise provided with a communication module, as described below, that is compatible with the communication module 430 as required to establish the communication link with the communication module 430 of the defibrillator. Together, the communication module of the defibrillator and the server, respectively, enable a communication link 472 to be established between the defibrillator processor 420 and the web server 410 for enabling the bidirectional flow of data between the defibrillator and web server 410.

In an illustrative embodiment, the communication module 430 may include a wireless module 432 and/or a network data connect module 434 as shown in FIG. 5. The wireless module may illustratively be a Wi-Fi module. Alternatively, the wireless module 432 may be a blue tooth module, a CDMA module, or any other communication module that enables a wireless communication link for the bidirectional flow of data between devices wirelessly. The network data connect module 434 may be a hardware and software based data connector configured to connect with a data outlet of the web server 410. The network data connect module 434 may be one or more ports and associated circuitry and software that allow bidirectional flow of data between the defibrillator processor 420 and the web server 410. Illustratively, the network data connect module is an Ethernet connector configured for connection to web server in a wired connection. Alternatively, the network data connect module may be an RS232 connector, a USB or other wire connector. Other connectors and hardware and software configurable for providing a wired connection between the communication module 430 and the web server 410 may be used for network data connect module 434 as are well known in the art.

Illustratively, the communication link 472 is based on the signaling system 7 (SS7) for controlling the communications between the defibrillator and the web server with telephone networks and VoIP systems.

The utilities 427 may be any hardware and/or software configured to provide data to or from the defibrillator. An illustrative utility 427 may be a parameter module which is a module configured to detect a parameter of a patient. The patient parameter may include one or more of the following measurements: a measurement of CO₂ exhaled by a patient; an electrical activity of the heart of a patient; an exchange of air between the lungs of a patient and the atmosphere; a pressure of the blood in a patient; a temperature of a patient; an oxygen saturation in the blood of a patient; a chest compression of a patient; an image of the internal structure of a patient; an oxygen saturation in the blood in the brain of a patient; the acidity or alkalinity of fluids in a patient; or other patient parameter.

The patient parameter of the CO₂ exhaled by a patient may be measured using capnography techniques. The patient parameter of the electrical activity of the heart of a patient may be measured using ECG techniques. The patient parameter of the exchange of air between the lungs of a patient and the atmosphere may be measured using ventilation techniques. The patient parameter of the measurement of the pressure of the blood in a patient may be measured using non-invasive blood pressure measurement techniques or invasive blood pressure measurement techniques. The patient parameter of the temperature of a patient may be measured using temperature measurement techniques. The patient parameter of the oxygen saturation in the blood of a patient may be measured using pulse oximeter techniques or tissue oximetry techniques. The patient parameter of the chest compression of a patient may be measured using chest compression detection and feedback techniques. The patient parameter of the image of the internal structure of a patient may be measured using ultrasound measurement techniques. The patient parameter of the oxygen saturation in the blood in the brain of a patient may be measured using cerebral oximetry techniques. The patient parameter of the acidity or alkalinity of fluids in a patient may be measured using non-invasive pH measurement techniques. These and other techniques and modules for generating the foregoing and other kind of patient parameter data for use with this disclosure are well known in the art.

Another illustrative utility may be an ECG data feed module configured to manage the feed of ECG data from an electrocardiogram (ECG) that may be tethered to the defibrillator. The utilities may include other data feeds modules configured to manage the feed of other data from within or from outside the defibrillator. A loudspeaker and a microphone may also be included in the defibrillator to enable a caregiver to communicate over communication link 472 with the web server 410, the server 460, and the clinical dashboard device 440 as described below.

Web server 410 may be one or more programmed computers that may be connected to the defibrillator 410 wirelessly or by wired connection in order to allow for the exchange of information between the defibrillator and the web server. Server 460 may be one or more programmed computers that may be connected to the web server 410 wirelessly or by wired connection in order to allow for the exchange of information between the server and the web server. The web server 410 has a processor 411, a memory 412, and a communication module 413 illustratively enabled with a WiFi communication module 414 and/or a network data communication module 415. Similarly, the server 460 has a processor 462, a memory 463, and a communication module 465 illustratively enabled with a WiFi communication module 466 and/or a network data communication module 467. The processor, memory, and communication module of each of web server 410 and server 460 are selected to handle the processing, memory, and communications tasks that are assigned to each server.

For example, the communication modules of each server may be configured to allow for the bidirectional flow of data between the server and another device. For example the communication module 413 of the web server 410 may illustratively be configured to be compatible with communication module 465 of server 460 and with the clinical dashboard device 440 as well as with the defibrillator 420 as previously described. The communication module of the web server 410 is configured to enable communication links 474 and 476 with the clinical dashboard device 440 and the server 460. This is additional to the communication link 472 that the web server 410 may establish with the defibrillator as previously described. The communication module of the server 460 is configured to enable communication link 476 with the web server 410.

As another example of the selection of the processor, memory, and communication module of each of web server 410 and server 460, the sizing of these components may also be determined by the processing tasks that may be assigned to each server. For example, each server may be any computer configured to serve the requests of client programs running on the same or other computers on a network. In FIG. 5, the computer of the server 460 may be a host computer configured to serve the requests of one or more client programs residing in the web server 410, the defibrillator 420, and/or the clinical dashboard 440. Similarly the computer of web server 410 may be a host computer serving as a web portal configured to provide a gateway for network terminals like the defibrillator and the clinical dashboard to communicate with server 460 and other servers that may be used in the system. The web server may serve the requests of one or more client programs residing in the server 460, the defibrillator 420, and/or the clinical dashboard device 440 in this example. Each of the web server and the server may serve yet other clients residing on it computer or some other computer to which the server may be connected. Depending on the computing service that the server is configured to offer, the server may include one or more of a file server for storing and making files accessible for reading and writing to the client, a print server that manages one or more printers, a network server that manages network traffic, a mail server that manages mail on a network, a database server that allows clients to interact with a database, and/or a hospital server for managing hospital records. The server may also be in communication with one or more other servers that themselves may include one or more of the foregoing or other servers. The sizing of the processor and memory components and the software requirements for each server includes these and other considerations.

The form factor of the computing device functioning as the server is illustratively a server but may also be a personal computer, a laptop computer, a tablet, a mobile computing device acting as a server. The servers may be provided with utilities which may include an adjunct medical device which may be a programmed computer that provides tools for monitoring the technique of a rescuer during the defibrillation process, such as applying CPR or proper positioning of the electrodes for application of a defibrillation charge on the patient. Illustratively, the device may monitor CPR chest compressions provided before or after defibrillation shock. For example, the device may measure the depth of a CPR chest compression, compare it to what it should be, and provide feedback to the user by way of instructions to go faster, deeper, etc. Alternatively, the adjunct medical device may be any other device that monitors defibrillation techniques and provides feedback to a rescuer at the site of the defibrillation.

The applications that the processors in either or both of web server 410 and server 460 may execute may also include existing applications that may be one or more software applications running on one or more computing devices external to the server.

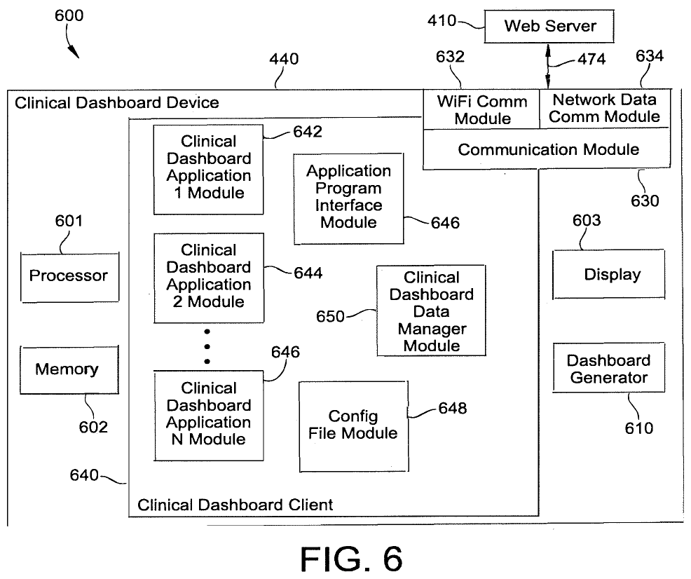

FIG. 6 is an enlarged view of an illustrative clinical dashboard device of the system in FIG. 4. The system in FIG. 6 is denoted as system 600. As shown in FIG. 6, the clinical dashboard device 440 comprises a processor 601, a memory unit 602, a display 603, a dashboard generator 610, a communication module 630, and a clinical dashboard client 640.

The processor 601 controls the functions of the clinical dashboard device 440. The processor 601 controls hardware and software residing in the clinical dashboard device 440. More specifically, the processor 601executes software illustratively residing in the memory unit 602 to operate the display 603, the dashboard generator 610, the communication module 630, and the clinical dashboard client 640.. The clinical dashboard device processor may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

The memory unit 602 of the clinical dashboard device can be any form of data storage device. It may be at least one of random access memory (RAM) and/or read only memory (ROM). Information can be stored permanently until overwritten and/or stored temporarily for use while the unit is active.

The display 603 may be a visual display capable of displaying data transmitted from processor 601. Displays for use with this disclosure may include an LCD screen, an e-paper display, or other bi-stable display, a CRT display or any other type of visual display.

Communication module 630 is hardware and software configured to transmit data to and from the clinical dashboard device 440. Illustratively, the communication module 630 is configured to transmit data from the defibrillator to the web server 410. Alternatively, the processor 601 may receive data from the web server through the communication module 630 to the clinical dashboard device. Hence, communication module 630 provides for the bidirectional transmission of data between the clinical dashboard device and the web server 410. In order to allow for the bidirectional flow of data between the clinical dashboard device and the external utility, the web server is likewise provided with a communication module, as previously described, that is compatible with the communication module 630 as required to establish the communication link with the communication module 630 of the clinical dashboard device. Together, the communication module of the clinical dashboard device 440 and the web server 410, respectively, enable the communication link 474 to be established between the clinical dashboard device 440 and the web server 410 for enabling the bidirectional flow of data between the clinical dashboard device and the web server 410.

In an illustrative embodiment, the communication module 630 may include a wireless module 632 and/or a network data connect module 634 as shown in FIG. 6. The wireless module may illustratively be a Wi-Fi module. Alternatively, the wireless module 632 may be a blue tooth module, a CDMA module, or any other communication module that enables a wireless communication link for the bidirectional flow of data between devices wirelessly. The network data connect module 634 may be a hardware and software based data connector configured to connect with a data outlet of the web server 410. The network data connect module 634 may be one or more ports and associated circuitry and software that allow bidirectional flow of data between the processor 601 and the web server 410. Illustratively, the network data connect module is an Ethernet connector configured for connection to web server in a wired connection. Alternatively, the network data connect module may be an RS232 connector, a USB or other wire connector. Other connectors and hardware and software configurable for providing a wired connection between the communication module 630 and the web server 410 may be used for network data connect module 634 as are well known in the art.

The processor, memory unit, and display of the clinical dashboard device may be combined into any form factor. Illustratively, the clinical dashboard device is in the form factor of a personal computer, a laptop computer, a tablet. But the clinical dashboard device may also take the form factor of a mobile computing device acting as a server. In one illustrative embodiment, the clinical dashboard device is a computer driven large screen monitor located in a hospital. In this case, the large screen monitor may be driven by a hospital computer, which may be a server.

The clinical dashboard client 640 is hardware and software configured to advantageously manage clinical dashboard data between one or more clinical dashboard application modules 642, 644, 646 and the server 410. The clinical dashboard client includes one or more clinical dashboard application modules 642, 644, 646, a clinical dashboard data manager module 650, a configuration file module 648, and an application program interface module 646.

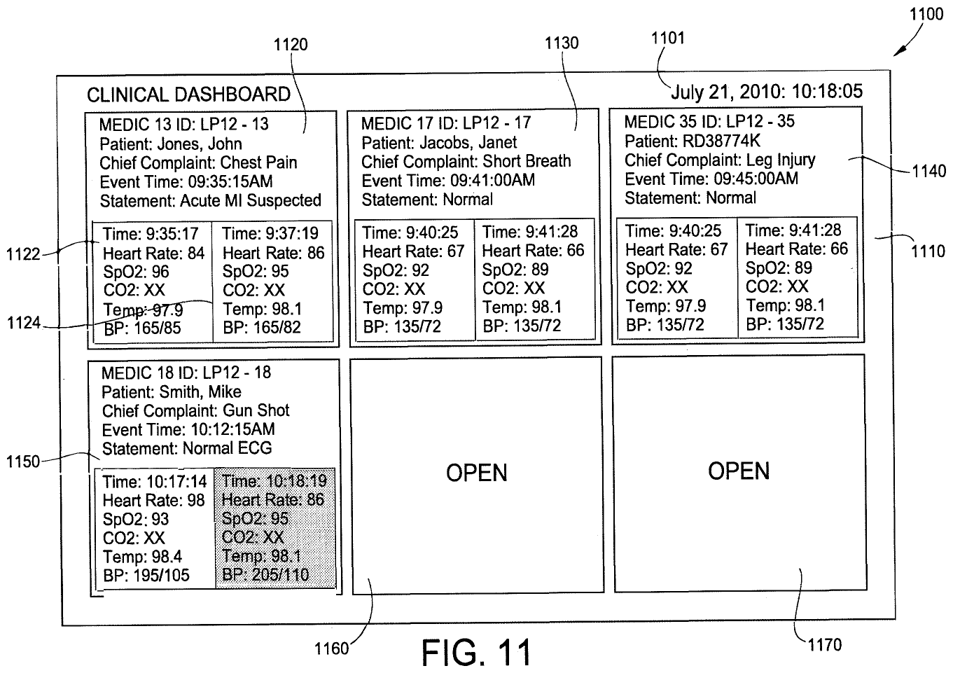

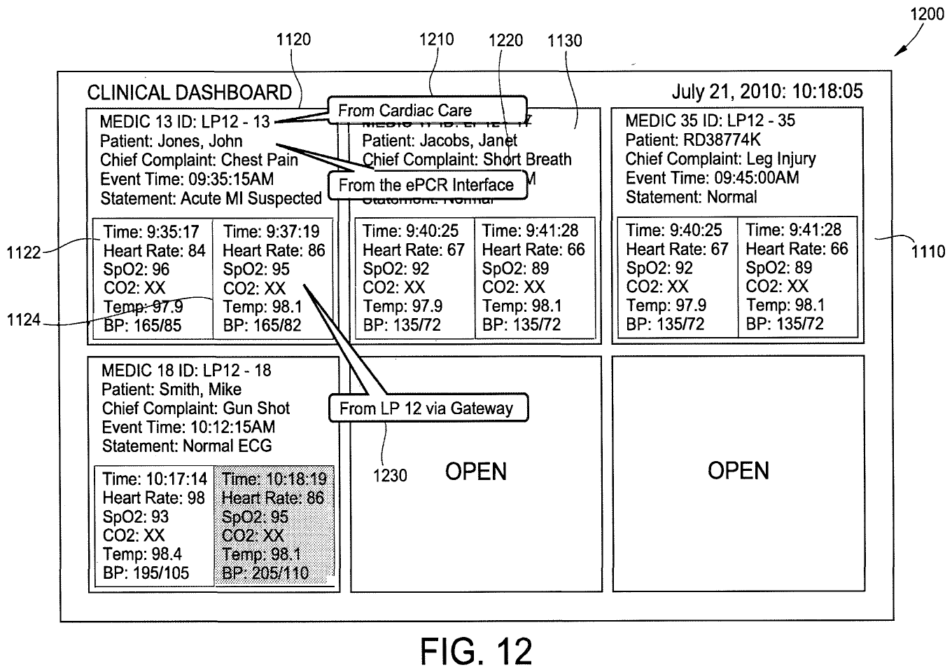

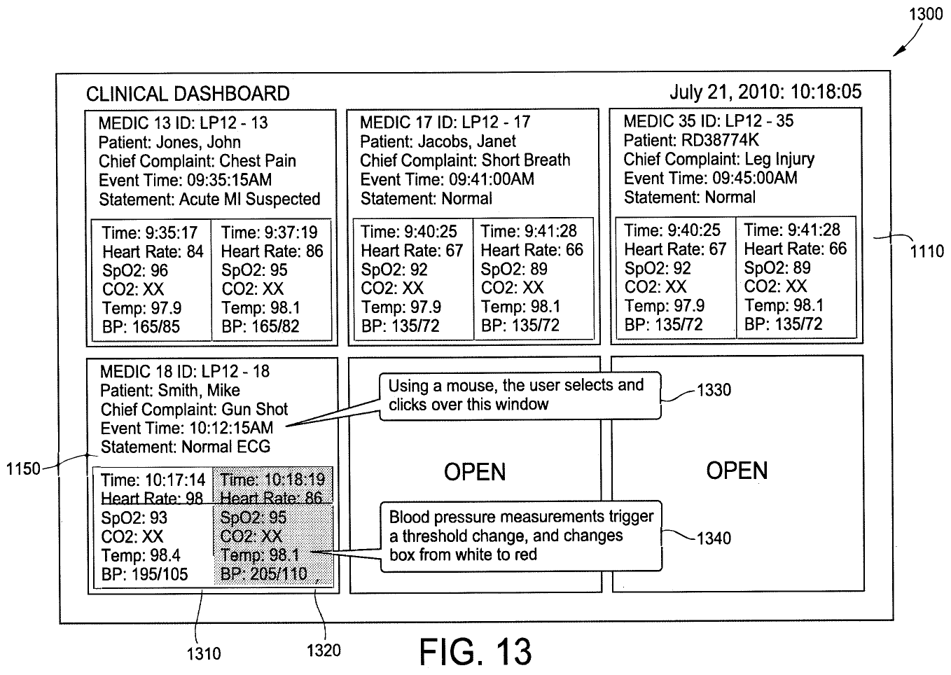

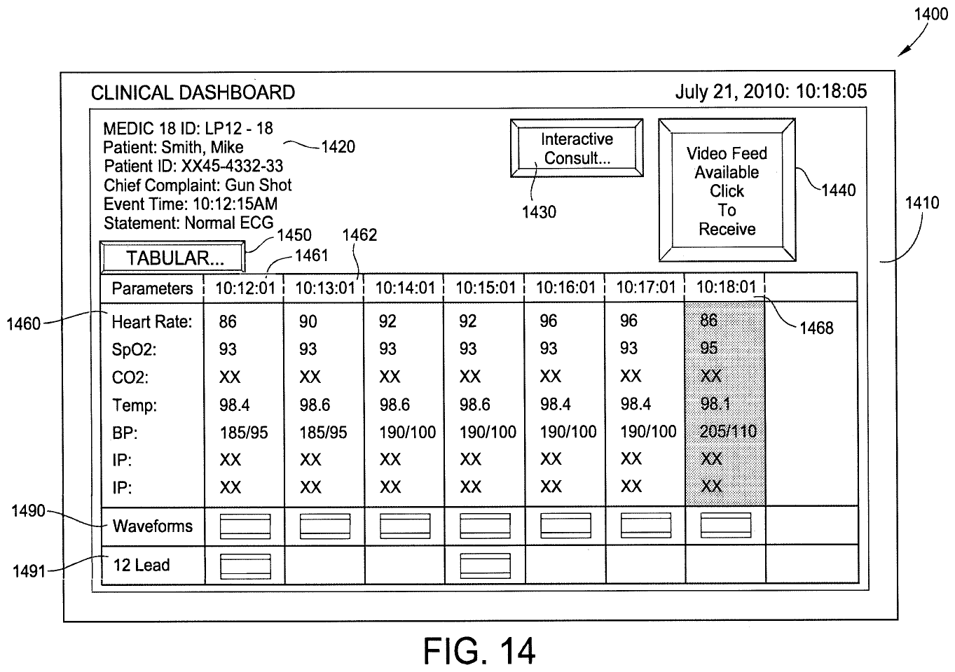

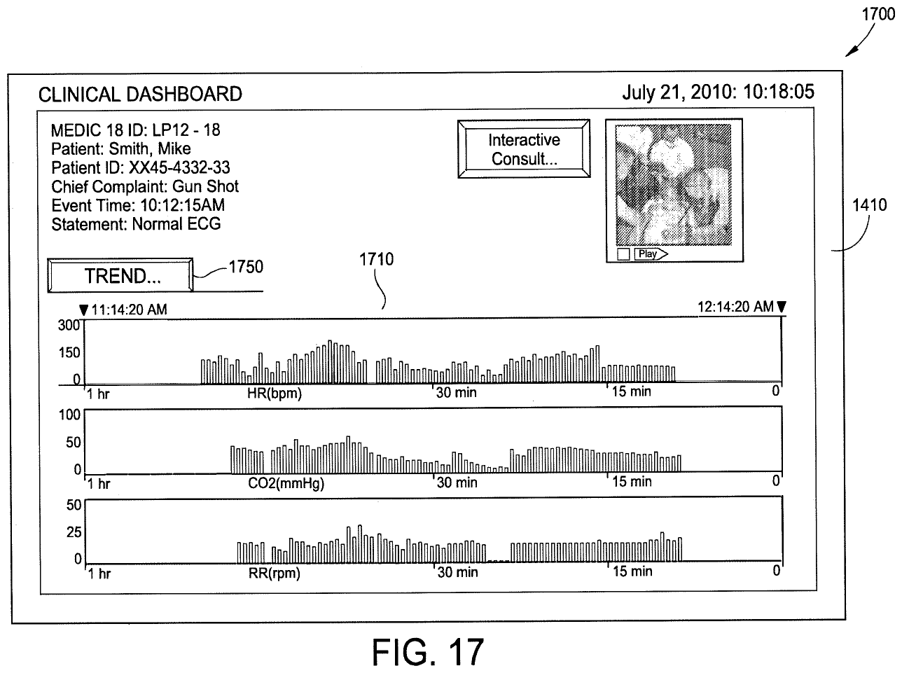

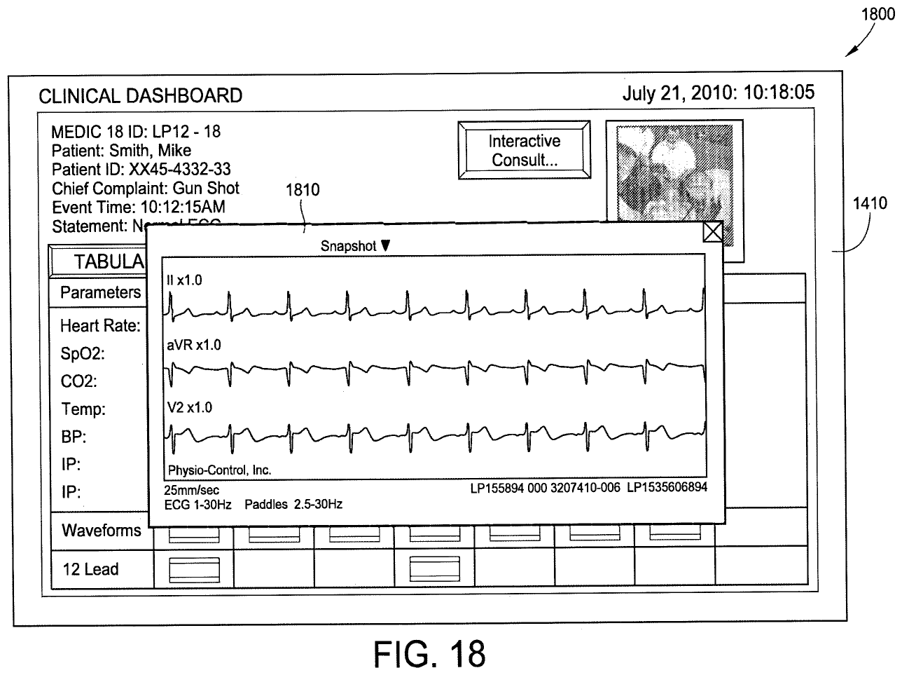

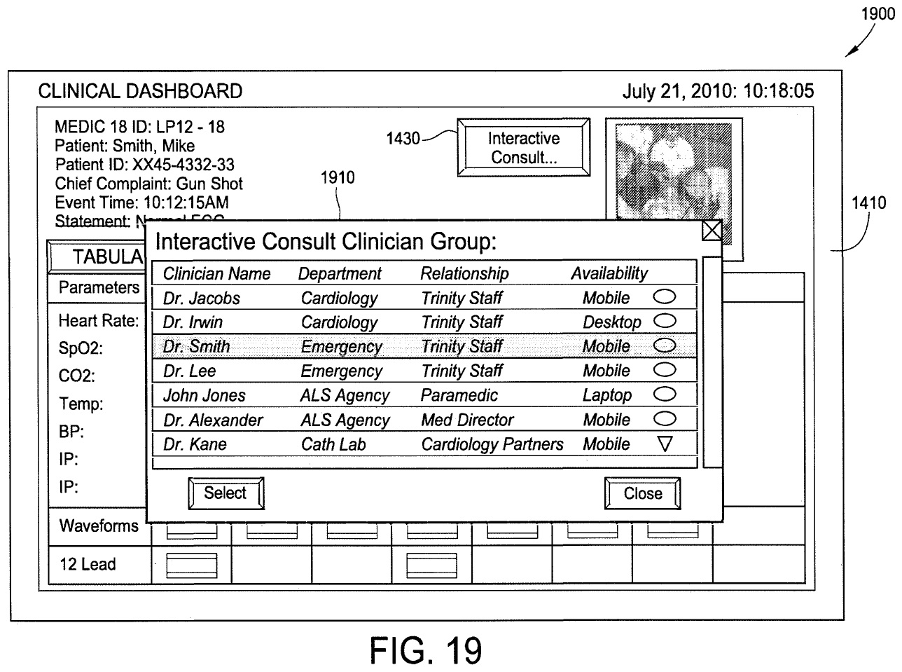

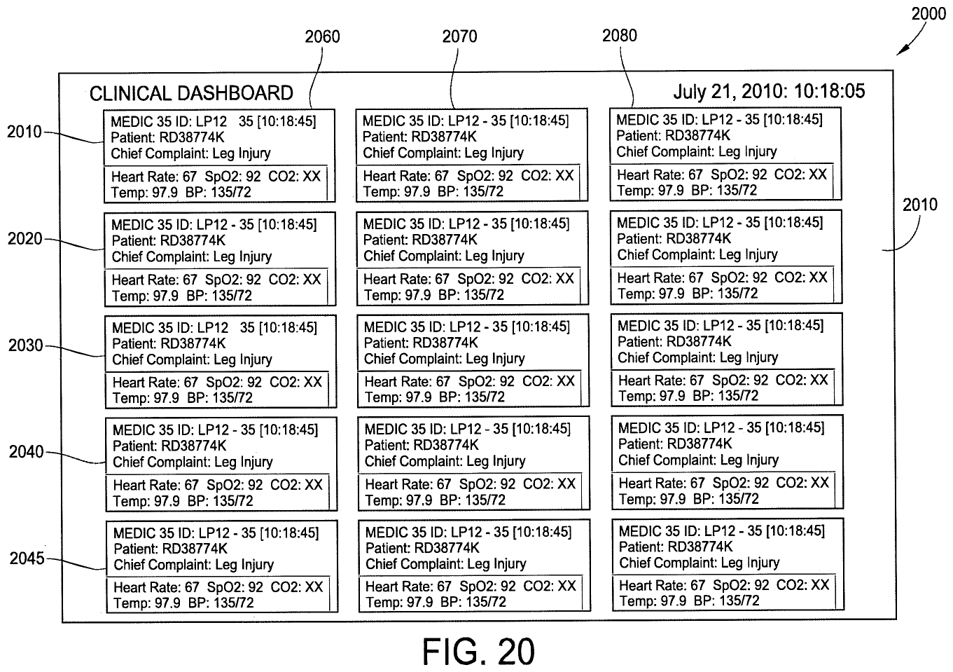

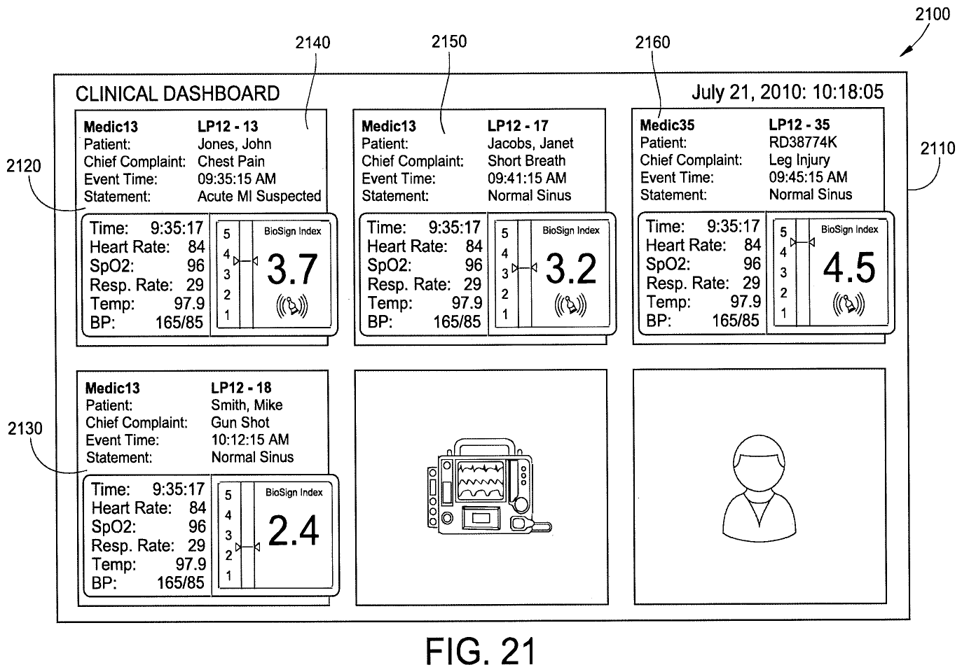

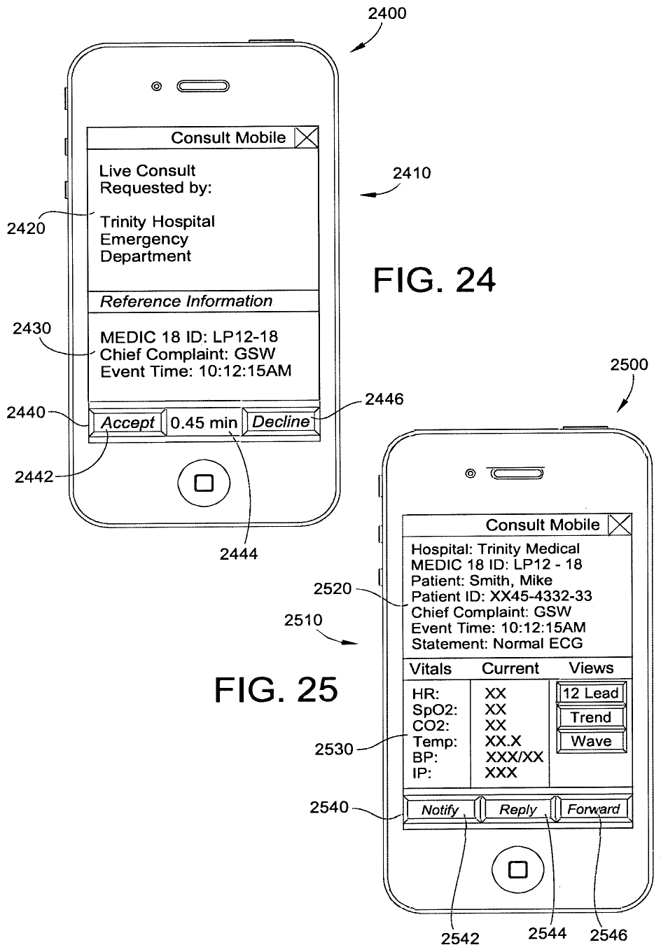

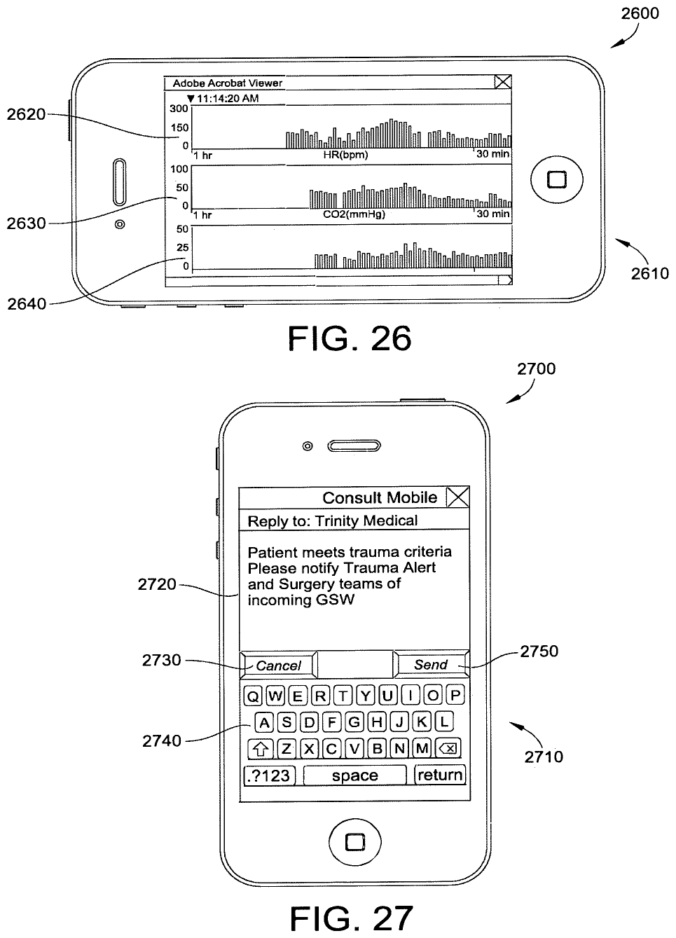

The clinical dashboard application modules 642, 644, 646 are hardware and software configured to package data in a predetermined manner for display on the display 603 of the clinical dashboard device. The input to each dashboard application module is clinical data that is provided by the web server 410 as described below. The dashboard application module processes that clinical data into a graphical format for display on the display 603 of the clinical dashboard device. FIGS. 11-27 illustrate different clinical dashboard data that the clinical dashboard device may display on it display. Illustratively, the clinical dashboard data may include patient data including vital data as shown in FIG. 11. The data may include data indicating the source of the patient data as shown in FIG. 12. The data may indicate threshold changes in a patient's condition as shown in FIG. 13. The data may include patient parameter data, interactive consultation, and video feeds as shown in FIGS. 14, 15, and 16. The data may include real time display of vital data over time as shown in FIG. 17. For example, the real time vital data may be an ECG as shown in FIG. 18. The data may include data pertaining to interactive consulting clinicians as shown in FIG. 19. The data may include summary chart record data on one or more patients as shown in FIG. 20. The data may be mixed and matched into a variety of graphical presentations as shown in FIGS. 21-23. The data may include background information on a patient as shown in FIGS. 24 and 25. The data may include comparative vital data as shown in FIG. 26. The data may include interactive messaging as shown in FIG. 27.

The data may be mixed and matched into a variety of graphical presentations. The clinical dashboard application modules are illustratively responsible for determining the graphical depictions of the clinical data that the clinical dashboard application module may request to depict. The clinical dashboard application modules may manipulate the content and presentation data in order to display the requested clinical data in a meaningful format according to this disclosure.

Other clinical dashboard data that the clinical dashboard device may display on it display may include other information such as decision points, decision reminders, possible etiologies, checklists, caregiver input, physiological data, trend line, timeline, time stamping, location, previous patient encounters, categories of data, timers that are set by data entry, etc. The dashboard may be further configured to display other information like check-off completions, predefined selections, care givers available, currently attending, or available, parameter or other information, coordination of data feeds from one or more inputs, defibrillator therapy activities, activities of partner systems, video procedures. For example, a video laryngoscope may provide tasks like apply the ventilator, check the heart rate, preview patient data. Configuration settings may be displayed such as user level, patient condition, the condition of the patient. The condition of the patient may be presented by display of patient data such as EMR, ePERS, and EMS. The tasks may display historic patient parameter profiles for the patient for comparison with current patient parameter profile. The tasks may display patient parameter profiles for a health patient for comparison with the parameter profile of the current patient.

The display of the data may be static or active. The clinical dashboard device creates the windows of static or active data as described below. The clinical dashboard device also creates the dependencies and links between the created windows displayed on the display 603 of the clinical dashboard device. This enables the clinical dashboard to display a set of clinical data that may be correlated. Through active links, a user may also interact with the displayed data and with other resources on the network. For example, a user may navigate forward or backward from a displayed data to a subsequent or previous window. The user may zoom in or out on a particular displayed data. The user may also interact in real or batch time with network resources including medical professionals for coaching. A user may interact in batch time with a library resource. A user may interact in real time with a medical professional through audio and/or audio/visual communications. A user may interact in delayed real time such as through text messaging. This disclosure provides other ways and one skilled in the art will appreciate still other ways in which a user may interact with the clinical dashboard displayed according to this disclosure.

It will be apparent to one skilled in the art that other forms of data, documents, images, video in any format may be downloaded or streamed from the web server 410 for use by the clinical dashboard application modules 642, 644, 646 in rendering meaningful graphical depictions for display to a caregiver in according to this disclosure.

The clinical dashboard client 640 includes the clinical dashboard data manager module 650. The clinical dashboard data manager module is hardware and software configured to to construct a dependency display of the graphical displays defined by the clinical dashboard application modules and to execute the dependency graph. Advantageously, the clinical dashboard data manager further includes an instance of a clinical dashboard data manager service that provides managed clinical dashboard data between one or more clinical dashboard applications in the clinical dashboard application modules and the server. The processor 601 of the clinical dashboard device is configured to execute the instance of the clinical dashboard data manager service to provide managed clinical dashboard data between the one or more clinical dashboard applications of the clinical dashboard application modules and the server.

The instance of clinical dashboard manager service configures hardware and software to accept data requests from a plurality of the clinical dashboard applications illustrated by clinical dashboard application modules 642, 644, 646 and for each data request, access the clinical dashboard data from the web server 410 for display on the display 603 of the clinical dashboard device. Illustratively, the clinical dashboard data manager module 650 is further configured to accept commands from the plurality of clinical dashboard application modules using a text-based mark-up language. In one embodiment, the text-based mark-up language is based on a standardized extensible markup language (XML). Alternatively, the clinical dashboard data manager service may be configured to accept commands from the one or more clinical dashboard applications in a text or binary format. The text-format can be any mark-up language such as XML or JSON.

The clinical dashboard data manager module is further configured to accept commands from the web server 410 using a text-based mark-up language. In one embodiment, the text-based mark-up language is based on a standardized extensible markup language (XML). Although a specific syntax based on XML has been described, it will be understood that any predefined syntax can be used to configure the clinical data manager service of the clinical dashboard data manager module. XML provides a convenient framework for a syntax, since it is a well-known standard for structured markup languages, and is readily extensible. Of course, xml is used only to structure the files, and the tags used, etc. are not part of a standard. But as previously explained, the language is not limited to XML. The clinical dashboard data manager service may be configured to accept commands from the one or more clinical dashboard applications in any text or binary format. The text-format can be any mark-up language such as XML or JSON.

As previously explained, the clinical data illustratively resides on the server 460 shown in FIGS. 4 and 5. Illustratively, the clinical dashboard device accesses the clinical data on the server 460 through the web server 410 which as previously described may serve as a gateway for accessing the server 460. Alternatively, the clinical data may reside on the web server 410.

The data request made by a clinical dashboard application for clinical data from the server may further contain a configuration file for configuring the clinical data manager service to the contents of a configuration file. The configuration files illustratively reside on the configuration file module 648 which is hardware and software directory of configuration files. In this way, the clinical dashboard data manager module may be configured to seamlessly receive graphs generated by the clinical dashboard applications. The clinical dashboard data manager modules manage those graphs and create dependencies between those graphs for display on the display screen of the display 603 of the clinical dashboard device. Advantageously, the clinical dashboard device 440 shown in FIG. 6 provides the editor for configuring the configuration file module 648 to the contents of a configuration file. For example, the clinical dashboard device 440 may define the protocols that the clinical dashboard data manager module 650 is to follow in managing the clinical display. The clinical dashboard device may further configure the clinical data residing on server 460 with protocols, rules, etc. for delivery and display on the clinical dashboard according to this disclosure.

The data request from the clinical dashboard application modules alone or in combination with a configuration file enables the clinical dashboard data manager service to establish a managed display of the clinical dashboard data from the server on the display of the clinical dashboard device according to the data request from the requesting clinical dashboard application. As part of this display management, the clinical dashboard device manager 650 may need to prioritize data requests from the same or different clinical application modules. Advantageously, the configuration file 648 may further provide the clinical dashboard data manager module with a directory of rules, preferences, and so on, for determining how and where to integrate a graph from a clinical dashboard application into one logical presentation of clinical dashboard data for display on the display of the clinical dashboard device using the teachings of this disclosure as explained below.

For instance, when a clinical dashboard application module is requesting the display of a protocol, a priority rule in the configuration file module 648 may provide that the protocol is to be displayed in the primary work area of the display screen. In this event, the protocol will displayed in the primary work area while the clinical data previously appearing in the primary work area will be moved to the background. As another example, a priority rule may provide that the protocol display is to be displayed in the primary working area except when any clinically critical information (e.g. AED Mode) is being displayed in the primary working area. In this event, the clinical dashboard data manager module may display the protocol as background in the primary work area.

In yet another illustrative example, the configuration file 648 provide the clinical dashboard data manager module with a directory of rules, preferences, and so on, that enable the clinical data manager service to parse the requests from the clinical dashboard applications based priority rules. For example, the clinical dashboard data manager module may establish a first display on the display screen requested by one clinical dashboard application based on a first priority rule, and establish a second display on the display screen requested by the same or another clinical dashboard application based on a second priority rule. In the immediately preceding example, the protocol graph was displayed as background in the primary work area because of the priority rule that required critical information to always be displayed in the foreground in the primary work area. This is like the first priority rule in the instant example. The instant example also provides a second priority rule that allows the display of the protocol in the foreground in a secondary work area in the event that critical information is being displayed in the primary work area. The second priority rule allows the display of the protocol as foreground information in a secondary work area of the display screen in this case.

The priority rules also allows the clinical dashboard data manager module to arbitrate between requests from competing clinical dashboard applications for clinical data from the server. For example, if one clinical dashboard application module makes a data request of the clinical dashboard data manager module for a predetermined feed of clinical data from the web server 410 and a second clinical dashboard application module makes a data request of the clinical dashboard data manager module for a feed of vital clinical data from the web server 410, the vital clinical data will be given the higher priority since the vital clinical data contains critical information for the caregiver.

The priority rules provide a way for the clinical dashboard data manager module to prioritize between competing requests from clinical dashboard applications for clinical data from the server and display of graphs generated by the applications. Hence, priority rules residing in configuration files in the configuration file module allow the clinical dashboard data manager module to arbitrate between two or more competing requests from clinical dashboard applications for data from the server and for display of graphical information on the display of the clinical dashboard device.

By routing all communications of the clinical dashboard applications through the clinical data manager module, the clinical dashboard data manager module is advantageously enabled to provide enhance management oversight over the flow and display of clinical data from the web server as requested by the clinical dashboard application modules of the clinical dashboard client.

The application program interface module 646 is hardware and software configured to interface the clinical dashboard application modules to the clinical dashboard data manager module.

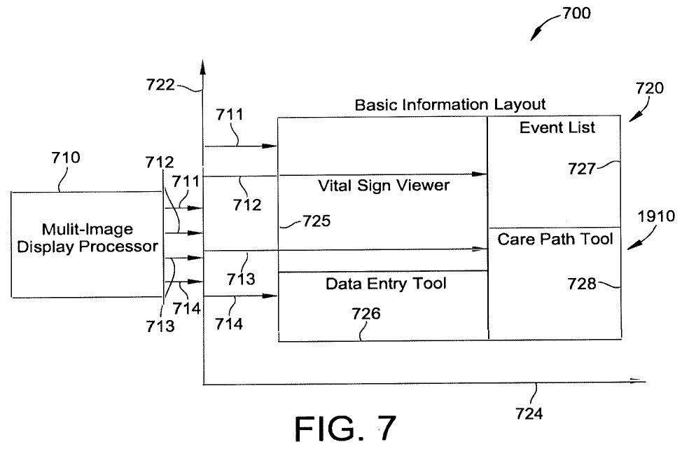

The dashboard generator 610 in FIG. 6 is hardware and software configured to provide a display of the dependency display of the graphical displays defined by the clinical dashboard application modules. FIG. 7 shows a portion of the clinical dashboard device 700 illustrating an illustrative dashboard generator 710 and a display screen 720 of a display. The output of the clinical dashboard data manager module 650 in FIG. 6 is the input to the dashboard generator 710 in this example. Alternatively the output may come directly from one or more of the clinical dashboard application modules. The dashboard generator treats the entire area of the screen 720 as a graphical canvas having x and y coordinates 724, 722, respectively, where windows can be created to tie the audio/video images coming as signal sources from the clinical dashboard applications module using clinical dashboard data provided by the web server to the processor of the clinical dashboard device. Illustrative audio/video images are shown in FIGS. 11-27 described below.

Display screen 720 shows the logical representation of how the screen is being treated by the display processor. Making a window on the canvas pertains to creating one logical video monitor, the size and shape of the screen determining the proportions of the image to be rendered and presented. As illustrated in FIG. 7, the display processor is driving the single screen with multiple windows created on the canvas. In the example, the processor is creating four windows on the screen—namely a vital sign viewer window 725, a data entry tool 726, an event list 727, and a care path tool 728. The processor shows the four signals that are driving these four windows as signal lines 711, 714, 712, and 713, respectively. The multiple display panels are unified to form a larger design working area. Alternatively, the window inputs and outputs from the display processor may be threaded as one logical input and output for rendering on the display.

As shown in FIGS. 11-27, a single picture can overlap on multiple screens. Once the display canvas is ready, windows of video, audio bar graph and other static text metadata can be inserted. Dynamic metadata may also be inserted. Any input sources coming into the processor can be assigned to any window created. The assignment is based upon the configuration of the clinical dashboard data manager module. As shown in FIGS. 11-27, multiple screens may be integrated into one logical presentation of clinical dashboard data using the teachings of this disclosure.

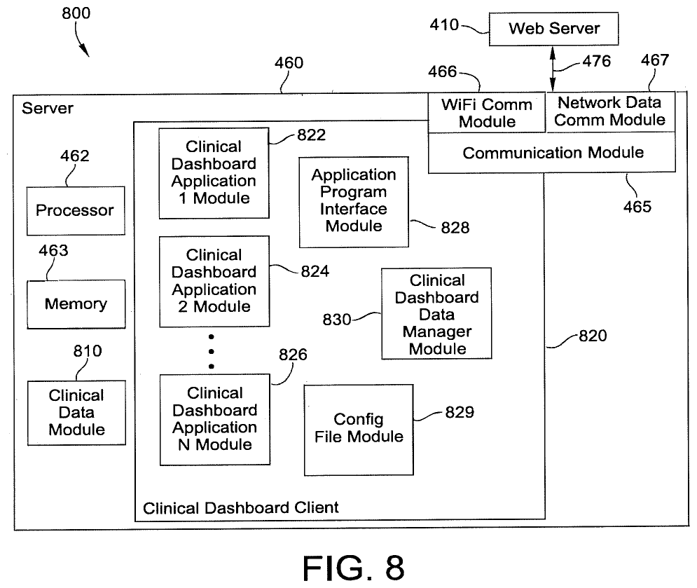

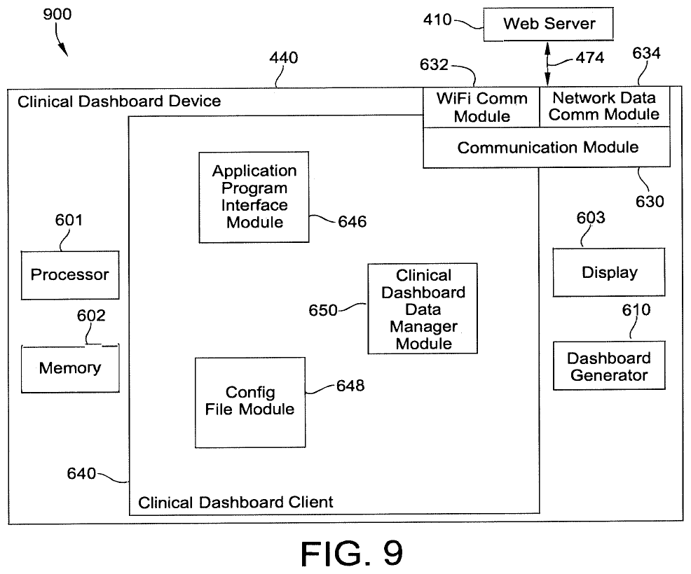

FIGS. 8 and 9 are an enlarged view of another illustrative clinical dashboard device and server of the system in FIG. 4. FIG. 8 shows a portion 800 of the system of FIG. 4 showing an enlargement of the server 460. The server comprises a processor 462, a memory 463 and a communication module 465 illustratively enabled with a WiFi communication module 466 and/or a network data communication module 467 as previously described in FIG. 5. The server further comprises a clinical dashboard module comprising clinical dashboard application modules 822, 824, 826; a clinical dashboard data manager module 830, an application program interface module 828, and a configuration file module 829. These components are similar to the like components described in connection with the clinical dashboard client 640 shown in FIG. 6 except that these components reside and operate at the server level.

For example, the clinical dashboard application modules 822, 824, 826 are hardware and software configured to package data in a predetermined manner for display on a display 603 of the clinical dashboard device shown in FIG. 9. The input to each dashboard application module is clinical data that is provided by the clinical data module 810 residing on the server in this example. The dashboard application module processes that clinical data into a graphical format for display on the display 603 of the clinical dashboard device shown in FIG. 9.

The clinical dashboard data manager module 830 is hardware and software configured to to construct a dependency display of the graphical displays defined by the clinical dashboard application modules and to execute the dependency graph. The clinical dashboard data manager includes an instance of a clinical dashboard data manager service that in the illustrative embodiment shown in FIG. 9 provides managed clinical dashboard data between the clinical data module 810 and the one or more clinical dashboard applications. The processor is configured to execute the instance of the clinical dashboard data manager service to provide managed clinical dashboard data between the clinical data module 810 and the one or more clinical dashboards. The instance of clinical dashboard manager service configures hardware and software to accept data requests from a plurality of the clinical dashboard applications 822, 824, 826 and for each data request, accesses the clinical dashboard data from the clinical data module 810 for display on the display 603 of the clinical dashboard device shown in FIG. 9.

The configuration files module 829 is hardware and software configured to specify a configuration for execution of one of the data requests requested by the clinical dashboard application modules. The configuration files module 480 illustratively provides the configurations for the data and data flow for each request that is made by the clinical dashboard applications. They also define priorities, restrictions, and so on as previously described in connection with clinical dashboard 638 in FIG. 6. The priority rules provide a way for the clinical dashboard data manager module to prioritize between competing requests from clinical dashboard applications for clinical data from the clinical data module and display of graphs generated by the applications.

The clinical dashboard device 440 shown in FIG. 9 is identical to the clinical dashboard device shown in FIG. 6 except that the clinical dashboard client 640 is operating in the embodiment of FIG. 9 without the clinical dashboard application modules which in the embodiment illustrated by FIGS. 8 and 9 reside on the server as illustrated in FIG. 8. The application program interface 646 in FIG. 9 is hardware and software configured to interface the clinical dashboard data manager module 650 with the clinical dashboard application modules residing on the server shown in FIG. 8. Further, in the embodiment shown in FIGS. 8 and 9, there are two clinical dashboard data manager modules—namely the clinical dashboard data manager module 650 residing in the clinical dashboard device shown in FIG. 9 and the clinical dashboard manager module 830 residing in the clinical dashboard module on the server. The distributed management of clinical data for display on a display screen of a clinical dashboard device requires the two modules to interface to each other in order to communicate with each other. The trade-off is enhanced clinical dashboard data management since the clinical dashboard data manager module in the clinical dashboard device may be customized to managing the clinical dashboard data at the clinical dashboard device level whereas the clinical dashboard manager module in the server may be customized manage the clinical dashboard data at the server level illustratively to optimize the delivery of clinical dashboard data residing in the server 460 to any one or more clinical dashboard devices that are serving as network terminals on the network.

Illustratively, the clinical dashboard data manager module of the clinical dashboard device 650 in FIG. 9 is further configured to accept commands from the web server 410 using a text-based mark-up language. In one embodiment, the text-based mark-up language is based on a standardized extensible markup language (XML). Additionally, the clinical dashboard data manager module 830 of the server in FIG. 8 is further configured to accept commands from the plurality of clinical dashboard application modules using a text-based mark-up language. In one embodiment, the text-based mark-up language is based on a standardized extensible markup language (XML). The clinical dashboard data manager module of the server may be further configured to communicate with the clinical dashboard data manager module of the clinical dashboard device using a text-based mark-up language. In one embodiment, the text-based mark-up language is based on a standardized extensible markup language (XML).

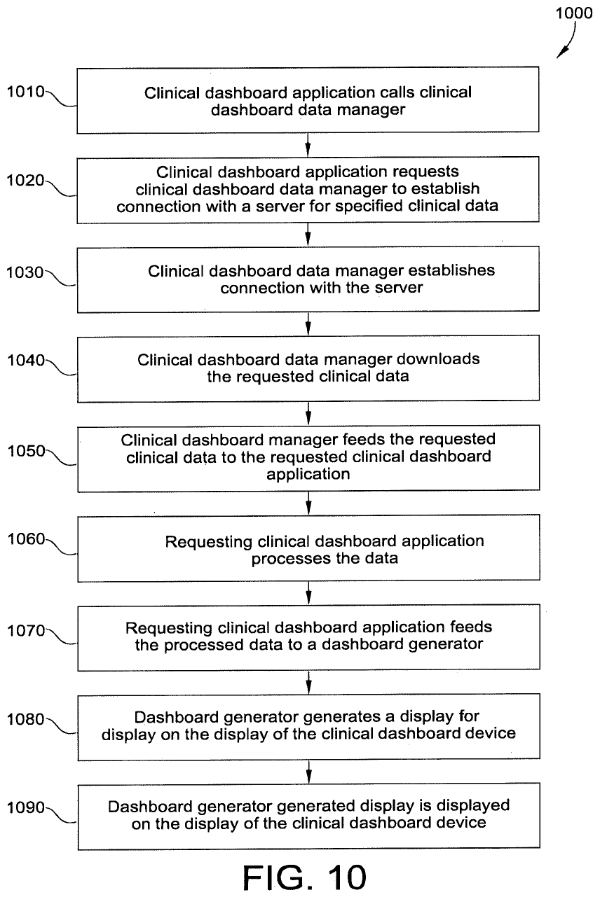

The operation of the decision support module 410 of FIG. 4 is illustratively shown in FIG. 10 as a flowchart 1000 for the generation of a clinical dashboard on a clinical dashboard device according to this disclosure. At step 1010, the clinical dashboard application calls the clinical dashboard data manager to secure clinical dashboard data from a server. At step 1020, the clinical dashboard application requests the clinical dashboard data manager to establish a connection with a server for a specified clinical data that the requesting clinical dashboard application wants to process. At step 1030, the clinical dashboard data manager establishes a connection with the server. At step 1040, the clinical dashboard data manager downloads the requested clinical data. At step 1050, the clinical dashboard manager feeds the requested clinical data to the requesting clinical dashboard application. At step 1060, the requesting clinical dashboard application processes the data. At step 1070, the requesting clinical dashboard application feeds the processed data under the management of the clinical dashboard data manager to a dashboard generator. At step 1080, the dashboard generator generates a display for display of the processed data on the display of the clinical dashboard device. At step 1090, the dashboard generator generates the display on the display of the clinical dashboard device.

FIGS. 11-26 show illustrative clinical dashboards configurable using the teachings of this disclosure.

FIG. 11 shows clinical dashboard display 1100 including patient data including vital patient data. The dashboard generator of this disclosure treats the entire area of the screen 1110 as a graphical canvas where windows 1120, 1130, 1140, 1150, 1160, and 1170 are created to tie the audio/video images coming as signal sources from the clinical dashboard applications module using clinical dashboard data provided by the web server to the processor. Windows 1160, 1170 are indicated to be inactive at the moment. In other words, windows 1160, 1170 are available to display addition patient records as needed.

Each of windows 1120, 1130, 1140, and 1150 includes a record that may identify the medic, the patient, the complaint, the event time, and a statement. Each of windows 1120, 1130, 1140, and 1150 are further provided with additional smaller sub-windows; although FIG. 11 shows only sub-windows 1122 and 1124 provided in window 1120. The sub-windows are used in this example to provide further records on the patient. In the embodiment of FIG. 11, the sub-windows are displaying vital patient data taken at a first point in time as recorded in sub-window 1122 and a second point in time as recorded in sub-window 1124. This allows two records on vital patient data taken at different times to be displayed; thereby showing how the vital patient data may have changed during the elapsed time which may provide useful coaching information to a caregiver.

The records displayed on the screen may be displayed as an active window. This allows the active window to be used to bring up additional records that may have been linked to the displayed window and to the identified patient. The active windows may also include audio/video images. More description of the active windows are provided below.

The clinical dashboard display 1100 further includes a date and time stamp 1101. The date and time stamp provides valuable information for time lining events for purposes of understanding progression of a condition and treatment as well as for historic and prospective documentation. The date and time stamping feature appears as a useful feature on many of the illustrative examples provided.

Advantageously, the clinical dashboard client of this disclosure provides a display of the dependency display of the graphical displays defined by the clinical dashboard application modules where windows are created to tie the audio/video images coming as signal sources from the clinical dashboard applications module using clinical dashboard data provided by the web server to the processor of the clinical dashboard device. The clinical dashboard client of this disclosure enables the logical representation in which the screen is treated by the dashboard generator. In the case of FIG. 11, six screens of windows 1120, 1130, 1140, 1150, 1160, 1170 may be integrated into one logical presentation of clinical dashboard data using the teachings of this disclosure.

FIG. 12 shows clinical dashboard display 1100 of FIG. 11 as clinical dashboard display 1200 including pop-up windows indicating from where a patient data was taken. The dashboard generator of this disclosure advantageously creates popup windows 1210, 1220, 1230 as single picture windows that overlap on multiple windows that are logically presented on the screen. For example the pop-up window 1210 overlaps windows 1120 and 1130. The pop-up indicates that the medic identified in window 1120 is from cardiac care. This disclosure thus allows one or more parts of the screen to be active at the same time that the parts of the screen may be displaying multiple windows. The active parts of the screen may be used by a caregiver to learn more about any data that is displayed in any window.

FIG. 13 illustrates further advantages possible by this disclosure from enabling portions of the screen to be active at the same time that the screen may be displaying multiple windows. FIG. 13 shows clinical dashboard display 1100 of FIG. 11 as clinical dashboard display 1300 including pop-up windows indicating from where a patient data was taken. Pop-up window 1330 enables a user to activate window 1150. Sub-window 1310, 1320 is also shown to be an active window in this example. In particular, the clinical data that is being displayed and updated in sub-window 1320 in real time is configured to provide an alert when the clinical data for blood pressure is above a threshold. In FIG. 13, the clinical dashboard provides this alert to the caregiver by way of a change in color of the sub-window 1320; although it will be appreciated by those skilled in the art that other visual or audible or other alerts may also be provided. The pop-up window 1340 provides yet another illustrative alert; in this case a textual alert to the caregiver that the blood pressure is above the threshold.

The threshold provides yet another illustrative example of a rule and/or priority configuration that may be provided by the configuration file module 648 of the clinical dashboard client 640 shown in FIG. 6 to enable the clinical dashboard data manager module 650 to manage the clinical dashboard display. In this example, the priority of sub-window 1320 was to display a white background unless the patient parameter reaches the threshold. In that event, the priority of sub-window 1320 is to display a red background; the color change providing an alert that the patient parameter has met or exceeds the trigger. In another example, the foregoing priority caused pop-up window 1340 to alert the caregiver of the condition. The threshold also provides an example showing how events within or outside of the clinical dashboard device may trigger another event. In the case of the color change, the patient parameter at a point in time is the event that triggered another event—namely, a color in color of the indicated window.

FIGS. 14-19 show clinical dashboard display 1100 of FIG. 11 as clinical dashboard displays 1400, 1500, 1600, 1700, 1800, and 1900 in FIGS. 14-19, respectively. These FIGS. further illustrate interactive features of this disclosure that provides enhanced coaching to a caregiver.

FIG. 14 shows a display screen 1410, a patient record window 1420, an interactive consulting active window 1430, an active video feed window 1440, an active tabular window 1450, and a window 1460 of patient vital data records 1461, 1462, and 1468, although other patient vital data records shown in FIG. 14 have not been numbered for this discussion. The patient vital data records 1461, 1462, and 1468 report patient data at different points in time. Each record also provides additional data such as the existence of data over time in the form of waveforms 1490 and the existence of 12 lead ECG data 1491. The record 1468 is also shown with a background that is presented in a color that is different from the background color of the other records appearing in window 1460. This indicates that the record 1468 contains patient data that is at or above a predetermined threshold; further illustrating the alert, priorities, and interdependence of active windows and events made possible by this disclosure.

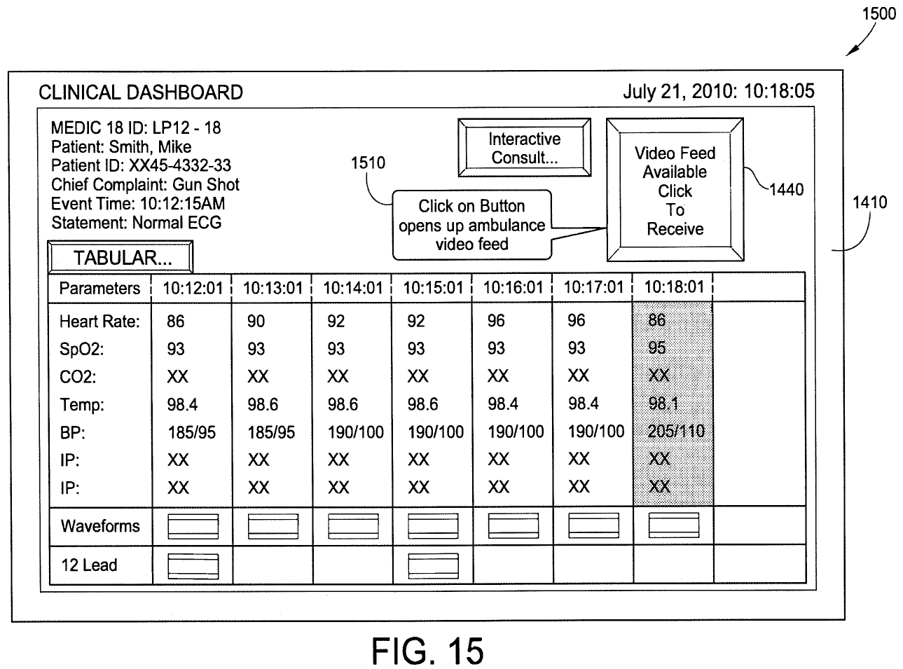

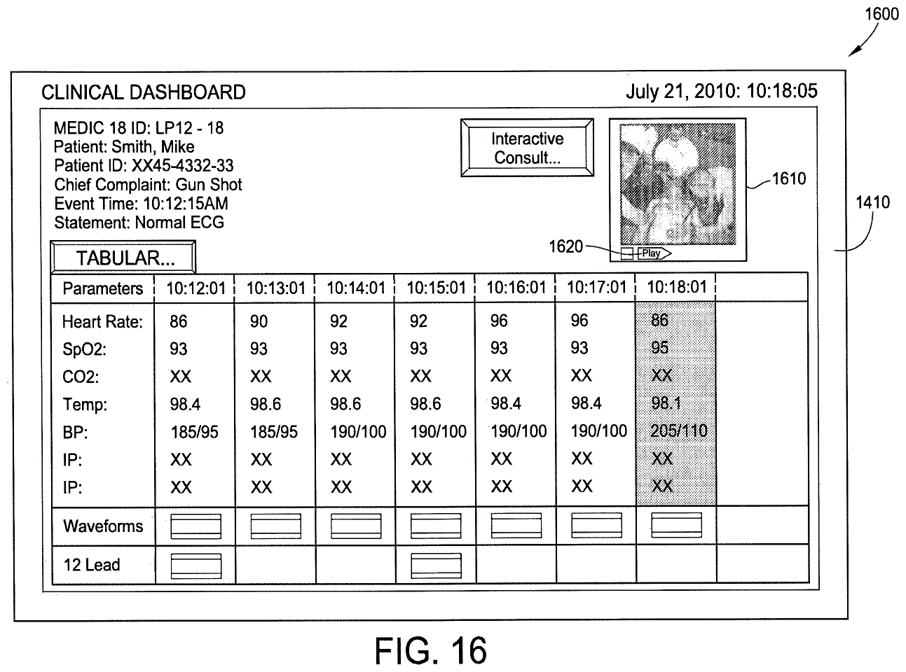

In FIG. 15, a pop-up 1510 alerts the caregiver to activate the active feed window 1440 for receipt of an active video feed relevant to a treatment. The active video feed is shown in FIG. 16 which appeared after the active video feed window was activated by the caregiver. Advantageously, the same window is used for both the hot button used to trigger the video feed and the video feed that the dashboard generator logically presents in the active video feed window 1440 under the management of the clinical dashboard data manager module. An active sub-window 1620 shown in FIG. 16 is also presented in the active video feed window to enable a user to control the play of the video feed.

On activation of the active video feed window 1440 shown in FIG. 14, the active tabular window 1450 shown in FIG. 14 is automatically replaced with an active trend window 1750 shown in FIG. 17. Activation of the active trend window 1750 causes a window of patient vital data to be displayed in window 1710. This feature further illustrates priorities, rules, etc. that may reside in the configuration file module 648 in FIG. 6 may determine the format of the clinical dashboard display of this disclosure. In this example, the display of window 1710 in FIG. 17 has a higher display priority than the display of window 1460 shown in FIG. 14. When the video feed window 1440 was activated, the higher priority display of the trend window 1750 and the display of patient vital data in window 1710 preempted the display of the tabular window 1450 and the patient parameter data displayed in window 1460. By assigning priorities to data, windows, portions of the work space, portions of the display screen, etc., the client dashboard device enables management of clinical dashboard content to provide the caregiver with needed information in a rapid, user friendly, insightful, and holistic way; thereby enhancing the coaching for the treatment of a patient.

In FIG. 18, critical ECG data is displayed in window 1810 as the caregiver is viewing the video feed. This allows the caregiver to monitor the ECG of the patient as the video is showing the patient being defibrillated at the site. This feature illustrates yet again how the client dashboard device of this disclosure brings highly relevant information directly to the caregiver for use in providing better coaching, documentation, historical recordation, and prospective education.

FIG. 19 shows a display of an active interactive consulting group window on the display screen. Advantageously, this window of patient vital data may be automatically displayed on activation of the active interactive consult window 1430. The interactive consult window 1430 provides a listing of clinicians who are actively linked to a communication link to enable the caregiver to contact the clinician on activation of the interactive consult window 1430. The interactive consult window 1430 also illustratively provides relevant information on each clinician which a caregiver may find useful in selecting the right care giver to contact. For example, if the caregiver is associated with Trinity, the caregiver may want to contact a member of the Trinity staff from the list for the consultation.

In FIG. 7 it was shown how display screen 720 shows the logical representation of how the screen is being treated by the display processor. Making a window on the canvas pertains to creating one logical video monitor, the size and shape of the screen determining the proportions of the image to be rendered and presented. As explained in FIG. 7, the display processor is driving the single screen with multiple windows created on the canvas. In the example shown in FIG. 7, the processor is creating four windows on the screen—namely a vital sign viewer window 725, a data entry tool 726, an event list 727, and a care path tool 728. In FIG. 20, the processor is creating a 5×3 panel of windows or 15 windows illustrated by the matrix of windows defined by the rows 2010, 2020, 2030, 2040, 2050 and columns 2060, 2070, and 2080 that define the matrix. Each window occupies a portion of display screen 2010 and may be used to define a unique record for use by the caregiver.

In the illustrative windows shown in FIG. 20, each window includes a record of a patient, a medic, a complaint, and certain vital data on the patient. Although FIG. 20 shows each of the records of data containing the same patient data, it will be appreciated that each record is illustratively different from each other in order to provided managed care over a broader set of patients. However, two or more of these windows may contain clinical data on the same patient in order to provide the caregiver with more in-depth information on a particular patient. In addition, the size and shape of each window may vary depending upon the clinical data and manner in which the data is designed to be displayed as previously illustrated in FIGS. 11-19. In this and other ways, this disclosure makes possible the presentation of a large set of records of data in an easy to read display of windows in which sub-windows may be created. In addition, each sub-window may be an active window which enables a user to activate still more windows of clinical data information or to zoom in or out on certain clinical data in order to create different perspectives on the clinical data. These features enable a caregiver to see and understand larger sets of data and their relationships more rapid, user friendly way. These and other active and inactive windows and sub-windows that may be nested and/or interconnected with other windows throughout the display screen made possible by the clinical dashboard device of this disclosure allows a caregiver to navigate through large amounts of clinical data rapidly for information required to perform a treatment. The clinical dashboard data manager module collects, aggregates, and correlates the graphical window depictions that are being rendered by the clinical dashboard application modules into one logical window for the dashboard generator to present as one logical window on the screen of the display. As previously explained, a caregiver has a very limited window of time to defibrillate a patient. The clinical dashboard device taught by this disclosure collects, aggregates, correlates, and displays large amounts of clinical data in a holistic way.

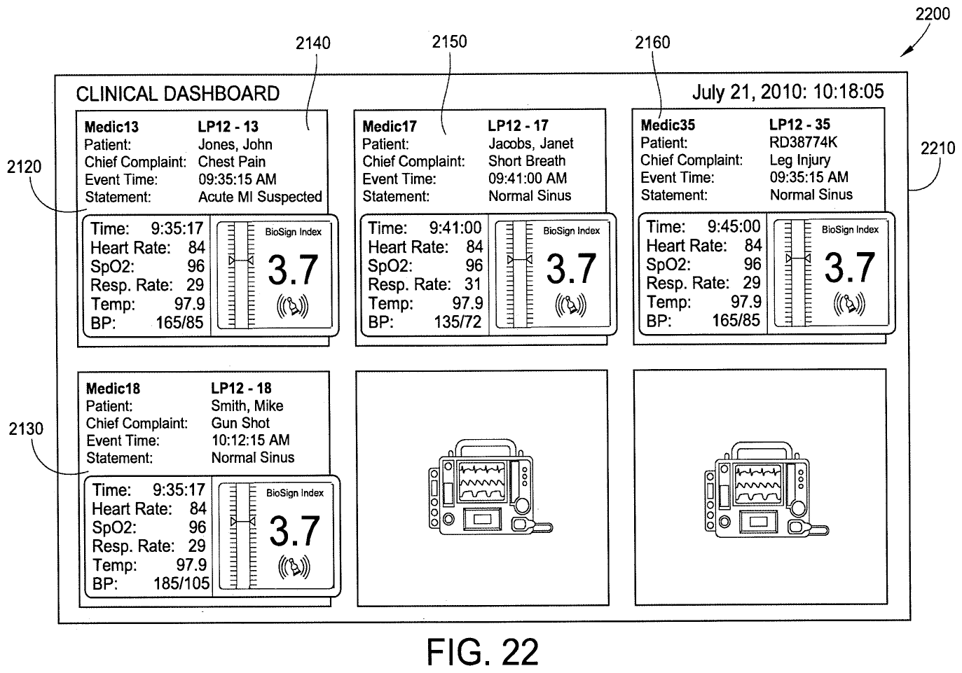

FIGS. 21-23 show alternative illustrative embodiments of display screens 2110, 2210, and 2310, respectively showing alternative logical representations of how a display screen of a clinical dashboard device of this disclosure may be treated by the clinical dashboard data manager module and dashboard generator of this disclosure. In FIGS. 21 and 22, the display processor is driving the single screen with multiple windows created on the canvas. In particular, the clinical dashboard device processor is creating six windows on the screen in this example. More particularly, the processor is presenting the windows on display screen 2110 as a 2×3 panel of windows or 6 windows illustrated by the matrix of windows defined by the rows 2120, 2130 and columns 2140, 2150, and 2160 that define the matrix. Each window occupies a portion of display screen 2110 and may be used to define a unique record for use by the caregiver.

In the illustrative windows shown in FIGS. 21 and 22, each of the windows occupying row 2120 and column 2140 includes a record on a different patient. In FIG. 21, the window at row and column number 2130/2150 is illustratively an active window dedicated to displaying clinical data from a defibrillator. The window at row and column number 2130/2160 is illustratively an active window dedicated to interactive consulting or for the display of individual patient specific clinical data or information. Hence, FIG. 21 illustrates a logical representation for treatment of the screen by the clinical dashboard data manager module and dashboard generator of this disclosure that enables a caregiver to monitor patent clinical data on four patients. The logical representation also allows the caregiver to use the remaining two windows to receive clinical data from a defibrillator or to enable interactive consulting or access of individual patient specific clinical data or information illustratively in connection with any one or more of the four patients being monitored in the logical representation presented on the display screen.

In FIG. 22, the windows at both row and column 2130/2150 and 2130/2160 are illustratively active windows dedicated to displaying clinical data from a defibrillator. Hence, FIG. 22 illustrates a logical representation for treatment of the screen by the clinical dashboard data manager module and dashboard generator of this disclosure that enables a caregiver to monitor patent clinical data on four patients. The logical representation also allows the caregiver to use the remaining two windows to receive clinical data from a defibrillator in connection with any one or more of the four patients being monitored in this logical representation presented on the display screen.

FIGS. 21 and 22 also show different ways in which the date and time information may be displayed on the display screen; each bringing this important data to the attention of the caregiver in different ways.

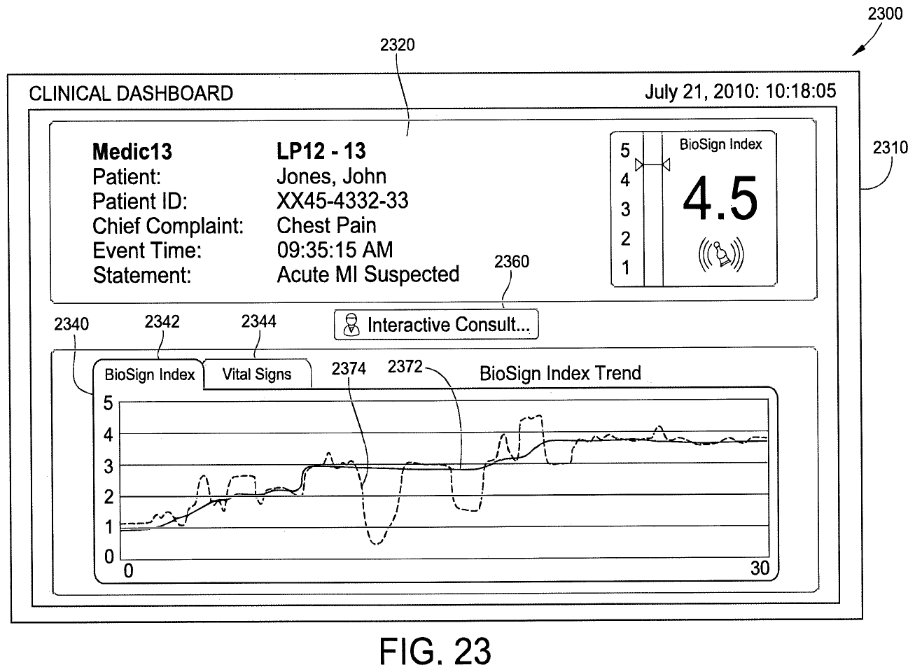

In FIG. 23, the display processor of this disclosure is driving a single screen 2310 with multiple windows created on the canvas. In particular, the processor is creating two windows on the screen—namely windows 2320, 2340. In this illustrative embodiment, window 2320 is dedicated to defining a unique record of patient data for use by the caregiver. The patient data may include patient identity, medic identity, complaint, event time, and statement of the problem. Window 2340 is advantageously configured as an active window that in this embodiment may be toggled back and forth between vital signs and biosigns by activating a vital signs tab 2344 and a biosign index tab 2342, respectively. When the vital signs tab 2344 is activated, the window 2340 displays vital signs data on a patient. Illustratively the vital signs window is linked to the patient record being displayed on window 2320 so that the vital signs that are being displayed in window 2340 are the vital signs of the patient identified in the patient record. When the biosign index is activated, the window 2340 displays a normal biosign index 2372 which is the biosign index displayed over time for a normal patient. The window 2340 also displays over the normal biosign index 2372 a patient biosign index 2374 which is the biosign index for the patient being treated.

The biosign index may be a software and hardware configuration of a construct for generating a predefined indice value associated with the overall health of the patient. The indice value represents a health score for a patient and various indice values may be generated depending upon what set of patient parameters a caregiver may want included in the indice value. For example, an indice value that is configured to provide a health score on blood pressure may be configured to display an indice value for the blood pressure of the patient illustratively based upon the height and weight and other physical properties of the patient. As another example, an indice value may combine blood pressure and respiratory rate readings into a combined health score of the patient in which case each reading would be weighted in the health score rating according to the algorithm. Once the biosign index tab is selected, the window 2340 displays a normal biosign index 2372 along with a biosign index 2374 for the patient being treated along a timeline which advantageously enables a caregiver to factor variations and trends discernible from the comparative biosign index data into the treatment of the patient.

As previously described, the clinical dashboard device is illustratively in the form factor of a personal computer, a laptop computer, a tablet. But the clinical dashboard device may also take the form factor of a mobile computing device acting as a server. The illustrative clinical dashboard presentations depicted in FIGS. 11-23 may illustrative be presented on the display of any of these or other form factors. In other words, the display on which the clinical dashboard of this disclosure may be presented may be a personal computer, a laptop computer, a tablet, a mobile computing device acting as a server, and so on. In one illustrative embodiment, the clinical dashboard device is a computer driven large screen monitor located in a hospital. In this case, the large screen monitor may be driven by a hospital computer, which may be a server.

FIGS. 24-27 illustrate the clinical dashboard presentations illustratively presented on the display of a mobile device, illustratively an iPhone®. In FIGS. 24 and 25, the clinical dashboard data manager module and the dashboard generator of this disclosure treat the entire area of the screen 2410, 2510 in FIGS. 24 and 25, respectively, as a graphical canvas where windows 2420, 2430, and 2440 are created in FIG. 24 and windows 2520, 2530, and 2540 are created in FIG. 25 to tie the audio/video images coming as signal sources from the clinical dashboard applications module using clinical dashboard data provided by the web server to the processor of the clinical dashboard device of this disclosure. In FIG. 24, window 2420 depicts a request for a live consultation. Window 2430 provides a record of the reference information. Window 2440 is presented with two active sub-windows 2442 and 2446 to enable a caregiver to accept or decline the requested consultation. Sub-window 2444 is an inactive sub-window in this example to indicate the time at which the consultation has been requested. Hence, the clinical dashboard of this disclosure provides presentation of clinical dashboard data in a cohesive, easy to interpret message, and which is interactive through the use of active windows that enable a user to interact with a message.

In FIG. 25, window 2520 depicts a record that is made available to the user in an interactive manner. More specifically, the clinical dashboard shown in FIG. 25 provides a window 2520 that provides a record of the reference information. Window 2530 provides vital clinical data on a patient. In this example, the vital clinical data is linked to the patient that is identified in the record displayed in window 2520. Window 2540 is presented with three active sub-windows 2542, 2544, and 2546 to enable a user to provide a notification, a reply, or to forward the record displayed in window 2520 and the vital clinical data displayed in 2530 to another resource such as another person, a network server, a computer of the user, etc. Hence, the clinical dashboard device of this disclosure provides presentation of clinical dashboard data in a cohesive, easy to interpret message, and which is interactive through the use of active windows that enable a user to interact with a message.

In FIGS. 26 and 27, the clinical dashboard data manager module and the dashboard generator of this disclosure treat the entire area of the screen 2610 and 2710, respectively, as a single window presented to tie the audio/video images coming as signal sources from the clinical dashboard applications module using clinical dashboard data provided by the web server to the processor of the clinical dashboard device. In FIG. 26, display screen 2610 shows a window presenting three waveforms of clinical data 2620, 2630, 2640. Hence, the clinical dashboard of this disclosure provides presentation of clinical dashboard data in a cohesive, easy to interpret display. In FIG. 27, display screen 2710 shows a window presenting a message 2720, a keyboard 2740, and active sub-windows 2730, 2750 that allow a user to cancel or send the message on to another resource. Hence, the clinical dashboard of this disclosure provides presentation of clinical dashboard data in a cohesive, easy to interpret message, and which is interactive through the use of active windows that enable a user to interact with a message.

There is thus disclosed a clinical dashboard device, system, method for providing clinical data for clinical dashboard display on a device is disclosed. The system includes a server for providing clinical dashboard data, which may include data from one or more defibrillators, and further includes a clinical dashboard device. The clinical dashboard device includes a processor, a communication module configured for enabling communication between the processor and the server for receiving the clinical dashboard data from the server, and a clinical dashboard client. The clinical dashboard client includes a clinical dashboard data manager module including an instance of a clinical dashboard data manager service that provides managed clinical dashboard data between one or more clinical dashboard applications and the server. The instance of the clinical dashboard data manager service further provides managed display of managed clinical dashboard data on the display of the clinical dashboard device. The processor is configured to execute the instance.

The clinical dashboard device of this disclosure may include a processor; a communication module configured for enabling communication between the processor and a server for receiving clinical dashboard data from the server; and a clinical dashboard client including a clinical dashboard data manager module including an instance of a clinical dashboard data manager service that provides managed clinical dashboard data between one or more clinical dashboard applications and the server. The processor is configured to execute the instance of the clinical dashboard data manager service to provide managed clinical dashboard data between the one or more clinical dashboard applications and the server.

The dashboard generator may be configured to provide a display of data generated by the one or more clinical dashboard applications; and a display for displaying the data provided by the dashboard generator. The instance of the clinical dashboard data manager service may further provide a managed display of managed clinical dashboard data on the display of the clinical dashboard device.

The clinical dashboard device may include a memory unit for storing the one or more clinical dashboard applications; and the instance of the clinical dashboard data manager service provides managed clinical dashboard data between the one or more clinical dashboard applications on the memory unit of the clinical dashboard device and the server. Alternatively, the one or more clinical dashboard applications may be stored on the server; and the instance of the clinical dashboard data manager service provides managed clinical dashboard data between the one or more clinical dashboard applications on the server and a data module configured for providing the clinical dashboard data on the server.

The configuration file module may be configured for defining priorities, rules, and the like for use by the clinical dashboard data manager module in managing the clinical dashboard data between the one or more clinical dashboard application and the server. The communication between the data server and the clinical dashboard device may be a network communication; and the data server may be a server connected to the network. The communication module may enable communication between the data server and the clinical dashboard device over a wireless or a wired connection. The clinical dashboard data manager service is configured to accept commands from the one or more clinical dashboard applications using a text-based mark-up language. The text-based mark-up language may be based on a standardized extensible markup language (XML).

A system for providing clinical data for clinical dashboard display on a device according to this disclosure may include the foregoing clinical dashboard display device; a server for providing clinical dashboard data, the server including a processor and a communication module configured for enabling communication between the processor and a defibrillator and a clinical dashboard device. The defibrillator includes a processor and a communication module configured for enabling communication between the processor and the server for providing defibrillator data to the server.

A method for displaying clinical dashboard data on a clinical dashboard device according to this disclosure includes accepting data requests from a plurality of clinical dashboard applications via a clinical data manager service of a clinical dashboard device; and for each data request, accessing clinical dashboard data from a server for display on a display of a clinical dashboard device. The data request may contain a configuration file for configuring the clinical data manager service to configure the clinical dashboard data in accordance with a configuration identified by the configuration file. The requested configuration configures the clinical dashboard data manager service to establish a display of the clinical dashboard data from the server on the display of the clinical dashboard device according to the requesting clinical dashboard application based on a priority rule. The requested configuration configuring the clinical data manager service may establish a first display on the display based on a first priority rule; and a second requested configuration configuring the clinical data manager service to establish a second display on the display based on a second priority rule. All communications of the clinical dashboard applications may be routed through the clinical data manager service.

The instructions that make up the foregoing and other methods of this disclosure may be stored on a computer readable medium which when executed by a processor causes the processor to perform the foregoing and other methods of this disclosure.

The clinical dashboard of this disclosure provides an integrated view of incoming patients to an Emergency Room from an Emergency Medical System (EMS). The clinical dashboard device may be a large screen monitor that can show patient information on multiple patients at once. All patients shown may be prior to their admittance into the Hospital or Emergency Department (ED). Patient information may be available via a wireless system that provides vital signs, waveforms, patient demographics, event information along with scene audio and video. Video can be from a scene or directed camera or from a diagnostic tool such as an Ultrasound and Video Laryngoscope. Smart alarms may be provided on top of the vital signs to provide an indication of overall patient condition (stable, unstable). Information from other sources such as Computer Aided Dispatch or Electronic Patient Care Reports may be included in the wireless information.

The clinical dashboard device of this disclosure allows patients to be tagged to what hospital is receiving the patient either by the paramedic in the field, a regional Patient Management System, or the Charge Nurse in the Emergency Room. The Charge Nurse can add additional priority information to help in the triage of patients as they arrive at the Emergency Department. Additional information could include the identification of the room/ area that the patient will be assigned to when they arrive.

In addition to determining which hospital is the receiving hospital, the system provides two way audio links to provide remote consultation by the regional Patient Management System staff or the Charge Nurse at the Emergency Department. One of the recommendations from the consultant function may be a request to use a particular decision support tool that may have been provided with the device. These decision support tools may take the form of a checklist and flow diagrams to help the caregiver perform the steps of a treatment according to a protocol and to ensure the integrity of the treatment process by enabling the caregiver to check off each step called for by the protocol when completed. The results of these actions may be available over the provided wireless link.

In addition to patients from the Emergency Medical System (EMS), the system may allow other pre-hospital patients to be monitored via the same system. Patients could be in areas like the ED Waiting Room, ED Triage Area, or ED Waiting areas. Patients could have some form or wireless monitor attached to them that would allow their information to be displayed on the clinical dashboard.

Through this disclosure, a clinical dashboard client is configured to advantageously manage clinical dashboard data between one or more clinical dashboard applications and the server. The clinical dashboard application modules package data in a predetermined manner for display on the display of the clinical dashboard device. The input to each dashboard application module is clinical data that is provided by the web server. The output from the clinical dashboard client drives a dashboard generator to illustratively create one logical video monitor containing multiple windows whose size and shape of the screen determine the proportions of the image to be rendered and presented. Windows of video, audio bar graph and other static text metadata, dynamic metadata, and other data can be inserted from any input sources coming into the processor which the clinical dashboard client can assign to any window created based upon the configuration of the clinical dashboard data manager module. Hence, the clinical dashboard of this disclosure provides presentation of clinical dashboard data through cohesive, easy to interpret windows, which may be active to enable user interaction. The clinical dashboard device of this disclosure enables the caregiver with a more holistic view of relevant data underlying a condition requiring treatment; thereby enabling the caregiver to make more informed treatment decisions.

In the method and system of this disclosure, the delivery and display of clinical data residing on servers throughout the network for clinical dashboard display on defibrillators throughout the network may be advantageously configured through protocols, rules, etc., by clinical dashboard devices in the network and managed by a clinical dashboard client in the defibrillators. For example, the clinical dashboard device illustratively may serve as the editor for configuring the client; programming the client in the defibrillator with the proper protocol, sequences, rules, configurations, etc. for the client to use in managing the clinical display The clinical dashboard device may further configure the clinical data residing on server 460 with protocols, rules, etc. for delivery and display on the clinical dashboard according to this disclosure.

In addition, for a given set of clinical dashboards, servers, and defibrillators that have been associated to each other through rules, permissions, etc. according to this disclosure, no matter where the clinical dashboard device, the defibrillator, or the server of the associated set may be on a network or internetwork, the clinical dashboard display has clinical data on associated servers configured by the associated clinical dashboard device and managed by the client on the defibrillator to provide a more robust, cohesive, and enhanced display to the user.

In addition, the system and method of this disclosure provide an integrated view of patient data from pre-hospital (ePCR electronic Patient Care Reporting), historical data from hospital records and the data from current monitoring of a patient. The system and method allow caregivers to selectively direct/route data from the pre-hospital to the appropriate receiving hospital, and allow hospitals to, based on review of the patient data, determine the appropriate care pathway for the patient and to redirect the patient, if necessary, to a more appropriate care location. The system and method provide an ability to monitor data from patients located in the pre-hospital transport environment, triage and emergency department; allowing for the ability to prioritize patient care for “sicker” patients. The system and method of this disclosure also provide the ability to enter “check-in” data, allowing patient data matching with existing hospital systems, (ability to retrieve patient data from previous incidents per item 1 above). These and other advantages are all made possible by the clinical dashboard of this disclosure.

In this description, numerous details have been set forth in order to provide a thorough understanding. In other instances, well-known features have not been described in detail in order to not obscure unnecessarily the description.

A person skilled in the art will be able to practice the present invention in view of this description, which is to be taken as a whole. The specific embodiments as disclosed and illustrated herein are not to be considered in a limiting sense. Indeed, it should be readily apparent to those skilled in the art that what is described herein may be modified in numerous ways. Such ways can include equivalents to what is described herein. In addition, the invention may be practiced in combination with other systems. The following claims define certain combinations and subcombinations of elements, features, steps, and/or functions, which are regarded as novel and non-obvious. Additional claims for other combinations and subcombinations may be presented in this or a related document.

1-32. (canceled)

33. A user monitoring device, comprising:
a communication module configured to receive user vital sign data from a remote user device; and
a processor configured to:
derive a vital sign parameter value from the user vital sign data;
determine that the vital sign parameter value is above a threshold value; and
generate an alert indicating that the vital sign parameter value is above the threshold value; and
output the alert to a caregiver.

34. The device of claim 33, wherein the user vital sign data includes a blood pressure of the user.

35. The device of claim 33, wherein the user vital sign data includes an electrocardiogram (ECG) signal of the user.

36. The device of claim 33, wherein the communication module is further configured to receive user vital sign data from a user parameter data device.

37. The device of claim 36, wherein the user parameter data device includes one or both of an electronic patient care record network or database, a rescuer input device, and a cardiac care device.

38. The device of claim 33, wherein the user vital sign data is measured at a single point in time.

39. The device of claim 33, wherein the user vital sign data is measured over a period of time.

40. The device of claim 33, wherein the processor is further configured to determine that the user parameter exceeds a threshold that a user is in need of emergency medical care.

41. The device of claim 33, wherein the user vital sign data includes a blood pressure of the user and the processor is further configured to determine that the blood pressure of the user indicates an acute cardiac event, a past cardiac event, or a pre-condition to an acute cardiac event.

42. The device of claim 33, wherein the user vital sign data includes an electrocardiogram (ECG) signal of the user, and the processor is further configured to determine that the ECG signal of the user indicates an acute cardiac event, a past cardiac event, or a pre-condition to an acute cardiac event.

43. The device of claim 33, wherein the user vital sign data includes one or both of a blood pressure of the user and an electrocardiogram (ECG) signal of the user, and wherein the processor is further configured to determine that one or both of the blood pressure and the ECG signal indicate an acute cardiac event.

44. The device of claim 33, wherein the alert includes one or more of a visual, audible, or haptic alert.

45. The device of claim 33, wherein the processor is further configured to transmit the alert to a remote caregiver device or network.

46. The device of claim 33, wherein the processor is further configured to transmit the alert to multiple remote caregiver devices or networks.

47. The device of claim 33, wherein the processor is further configured to output the alert on the user monitoring device.

48. The device of claim 33, wherein the remote user device includes a medical device configured to deliver defibrillation shock to the user.

49. The device of claim 33, wherein the remote user device includes one or more vital sign sensors configured to sense respective one or more vital signs of the user.

50. The device of claim 33, wherein the processor is further configured to establish a communication channel between the remote user device and the user monitoring device after the processor determines that the vital sign parameter value is above the threshold value.

51. The device of claim 50, wherein the processor is further configured to output a prompt to the caregiver to begin an interactive consulting communication session with the remote user device.

52. The device of claim 51, wherein the processor is further configured to receive input from the caregiver to accept the interactive consulting communication session with the remote user device before beginning the interactive consulting communication session.

53. The device of claim 52, wherein the processor is further configured to output an instruction to begin a live video feed between the remote user device and the user monitoring device in response to the input from the caregiver to accept the interactive consulting communication.

54. The device of claim 50, wherein processor is further configured to:
determine that the vital sign parameter value indicates an acute cardiac event;
establish an interactive consulting communication session between the remote user device and the user monitoring device; and
transmit caregiver coaching data to the remote user device using the interactive consulting communication session.

55. A patient monitoring device, comprising:
a communication module configured to receive one or both of a blood pressure and an electrocardiogram (ECG) signal from a remote patient device; and
a processor configured to:
analyze the one or both of the blood pressure and the ECG signal to determine a patient parameter value for one or both of the blood pressure and the ECG signal; and
generate an alert indicating that the patient parameter value is above a threshold value associated with an acute cardiac event; and
output the alert to a caregiver.
